(19) United States
(12) Patent Application Publication
(10) Pub. No.: US 2024/0218075 A1
(43) Pub. Date: Jul. 4, 2024

(54) USE OF CHIMERIC ANTIGEN RECEPTOR MODIFIED CELLS TO TREAT CANCER

(71) Applicant: Innovative Cellular Therapeutics Holdings, Ltd., George Town (KY)

(72) Inventors: Lei Xiao, Rockville, MD (US); Chengfei Pu, Shanghai (CN); Zhiyuan Cao, Shanghai (CN); He Sun, Shanghai (CN); Mao Bi, Shanghai (CN); Zhao Wu, Shanghai (CN)

(73) Assignee: Innovative Cellular Therapeutics Holdings, Ltd., George Town (KY)

(21) Appl. No.: 18/439,435

(22) Filed: Feb. 12, 2024

Related U.S. Application Data
(63) Continuation of application No. 16/997,553, filed on Aug. 19, 2020, now Pat. No. 11,932,700.
Application No. 16/997,553 is a continuation of application No. 15/942,112, filed on Mar. 30, 2018, now Pat. No. 10,793,638.
Application No. 15/942,112 is a continuation of application No. 15/685,670, filed on Aug. 24, 2017, now Pat. No. 9,932,405.
Application No. 15/685,670 is a continuation-in-part of application No. PCT/CN2017/078740, filed on Mar. 30, 2017.
(60) Provisional application No. 62/317,261, filed on Apr. 1, 2016.

(51) Int. Cl. C07K 16/28; A61K 35/17; A61K 38/17; A61K 39/00; A61K 39/395; C07K 14/47; C07K 14/705; C07K 14/725; C12N 5/0783; C12N 7/00

(52) U.S. Cl. CPC C07K 16/2896; A61K 35/17; A61K 38/1774; A61K 39/00; A61K 39/39558; C07K 14/4748; C07K 14/705; C07K 14/7051; C12N 5/0636; A61K 2039/505; A61K 2039/5158; A61K 2039/585; C07K 2319/33; C12N 7/00; C12N 2501/515

(57) ABSTRACT

The present disclosure relates to compositions and methods for compositions, methods, and kits for treating cancer using chimeric antigen receptor (CAR) modified cells. Some embodiments of the present disclosure relate to an isolated nucleic acid sequence encoding CAR. The CAR may include an antigen binding domain, a transmembrane domain, a costimulatory signaling region, and a CD3 zeta signaling domain. The antigen binding domain may bind to an antigen of a non-essential organ.

### CROSS REFERENCE TO RELATED APPLICATIONS

This application is a continuation of co-pending U.S. patent application Ser. No. 16/997,553 filed Aug. 19, 2020, which is a continuation of U.S. patent application Ser. No. 15/942,112 filed Mar. 30, 2018, now U.S. Pat. No. 10,793,638, which is a continuation of U.S. patent application Ser. No. 15/685,670 filed Aug. 24, 2017, now U.S. Pat. No. 9,932,405, which is a continuation-in-part of International application number PCT/CN2017/078740, filed Mar. 30, 2017, titled “Use of chimeric antigen receptor modified cells to treat cancer,” which claims priority to U.S. Provisional Patent Application No. 62/317,261, filed Apr. 1, 2016, entitled “Use of chimeric antigen receptor modified cells to treat cancer,” which are hereby incorporated by reference in their entirety.

### SEQUENCE LISTING

A computer readable XML file, entitled “1071-0028USC4_Sequence Listing-ST26.xml,” created on or about Feb. 1, 2024, with a file size of about 78,611 Bytes, contains the sequence listing for this application and is hereby incorporated by reference in its entirety.

### TECHNICAL FIELD

The present disclosure relates to modified cells and uses, in particular to compositions and methods for treating cancer using chimeric antigen receptor (CAR) modified cells.

### BACKGROUND

Cancer is known as malignant tumors involving abnormal cell growth with the potential to invade or spread to other parts of the body. In humans, there are more than one hundred kinds of cancer, for example, breast cancer occurred in epithelial tissue of the breast. Since breast cancer cells lose characteristics of normal cells, the connection between breast cancer cells is loose. Once the cancer cells are exfoliated, these exfoliated cancer cells spread over bodies via the blood or lymph systems and therefore become life-threatening. Currently, breast cancer has become one of the common threats to women's physical and mental health. Immunotherapy (e.g., CAR T) has been proved to be effective for treating cancer. But there is a need to improve the immunotherapy to be more effective for certain cancer such as solid tumors.

### SUMMARY

Embodiments of the present disclosure relate to compositions, methods, and kits for treating cancer using chimeric antigen receptor (CAR) modified cells.

Some embodiments of the present disclosure relate to an isolated nucleic acid sequence encoding a CAR. The CAR may include an antigen binding domain, a transmembrane domain, a costimulatory signaling region, and a CD3 zeta signaling domain. The antigen binding domain may bind to an antigen of a non-essential organ. For example, the antigen binding domain binds to an antigen that is expressed on the surface of a non-essential organ cell present in a microenvironment of a tumor.

Some embodiments of the present disclosure relate to an isolated CAR including an antigen binding domain, a transmembrane domain, a costimulatory signaling region, and a CD3 zeta signaling domain. The antigen binding domain may bind to an antigen of a non-essential organ.

Some embodiments of the present disclosure relate to a pharmaceutical composition including human T cells. The human T cells may include a nucleic acid sequence encoding a CAR. The CAR may include an antigen binding domain, a transmembrane domain, a costimulatory signaling region, and a CD3 zeta signaling domain. The antigen binding domain may bind to an antigen of a non-essential organ.

Some embodiments of the present disclosure relate to a cell including a nucleic acid sequence encoding a CAR. The CAR may include an antigen binding domain, a transmembrane domain, a costimulatory signaling region, and a CD3 zeta signaling domain. The antigen binding domain binds to an antigen of a non-essential organ. For example, the cell is selected from the group consisting of a T cell, a natural killer (NK) cell, a cytotoxic T lymphocyte (CTL), and a regulatory T cell.

Some embodiments of the present disclosure relate to a vector comprising a nucleic acid sequence encoding a CAR. The CAR may include an antigen binding domain, a transmembrane domain, a costimulatory signaling region, and a CD3 zeta signaling domain. The antigen binding domain may bind to an antigen of a non-essential organ.

Some embodiments of the present disclosure relate to a method for stimulating a T cell-mediated immune response to a cell population in a non-essential organ of a subject. The method may include administering to a subject an effective amount of a cell genetically modified to express a CAR. The CAR may include an antigen binding domain, a transmembrane domain, a costimulatory signaling region, and a CD3 zeta signaling domain. The antigen binding domain is selected to recognize the cell population of the non-essential organ specifically.

Some embodiments of the present disclosure relate to a method of treating a subject with cancer. The method may include administering to the subject a cell genetically engineered to express a CAR. The CAR may include an antigen binding domain, a transmembrane domain, a costimulatory signaling region, and a CD3 zeta signaling domain. The antigen binding domain may bind to an antigen of a non-essential organ. For example, the cell is selected from the group consisting of a T cell, a natural killer (NK) cell, a cytotoxic T lymphocyte (CTL), and a regulatory T cell.

In some embodiments, the antigen binding domain is an antibody, an antigen-binding fragment thereof, or a ligand thereof. For example, the antigen-binding fragment is a Fab or a scFv.

In some embodiments, the antigen is expressed on a non-essential organ cell present in a microenvironment of a tumor.

In some embodiments, the tumor is a breast cancer. In certain embodiments, the antigen is a mammary gland antigen. For example, the mammary gland antigen is prolactin receptor (PRLR) having SEQ ID NO: 29. In some embodiments, the antigen binding domain is prolactin receptor ligand having SEQ ID NO: 20 or 44.

In some embodiments, the tumor is colorectal cancer. In certain embodiments, the antigen is a colon antigen. For example, the colon antigen is Guanylate cyclase 2C (GUCY2C) having SEQ ID NO: 33.

In some embodiments, the tumor is gastric cancer. In certain embodiments, the antigen is a gastric gland antigen. For example, the gastric gland antigen is Mucin 17 (Muc17) having SEQ ID NO: 31.

In some embodiments, the tumor is a bladder cancer. In certain embodiments, the antigen is a bladder antigen. For example, the bladder antigen is CD207 having SEQ ID NO: 35.

In some embodiments, the tumor is an ovary tumor. In certain embodiments, the antigen is an ovary antigen. For example, the ovary antigen is Frizzled family receptor 10 (FZD10) having SEQ ID NO: 25.

In some embodiments, the tumor is a thyroid tumor. In certain embodiments, the antigen is a thyroid antigen. For example, the thyroid antigen is Thyroid stimulating hormone receptor (TSHR) having SEQ ID NO: 27.

In some embodiments, the costimulatory signaling region may include the intracellular domain of a costimulatory molecule selected from the group consisting of CD27, CD28, 4-IBB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

In some embodiments, the antigen binding domain may include at least one of SEQ ID NOs: 2-20 or 44.

In some embodiments, the antigen binding domain may include SEQ ID NO: 3 or 4, or a combination thereof, and the tumor is ovary tumor.

In some embodiments, the antigen binding domain may include SEQ ID NO: 6 or 7, or a combination thereof, and the tumor is thyroid tumor.

In some embodiments, the antigen binding domain may include SEQ ID NO: 9 or 10, or a combination thereof, and the tumor is breast cancer.

In some embodiments, the antigen binding domain may include SEQ ID NO: 12 or 13, or a combination thereof, and the tumor is gastric cancer.

In some embodiments, the antigen binding domain may include SEQ ID NO: 15 or 16, or a combination thereof, and the tumor is colorectal cancer.

In some embodiments, the antigen binding domain may include SEQ ID NO: 18 or 19, or a combination thereof, and the tumor is bladder cancer.

In some embodiments, the antigen binding domain may include SEQ ID NO: 20 or 44, and the tumor is breast cancer.

Some embodiments of the present disclosure relate to a method of selecting an antigen binding domain for a CAR for treating a subject with tumor cells. The method may include determining an organ of cells from that the tumor cells derived, determining that the organ is a non-essential organ with respect to the subject, searching a database to identify multiple markers that are expressed in a cell population of the organ, selecting a marker of the multiple markers based on a predetermined condition, and generating cells comprising a CAR using cells from the subject. The CAR may include an antigen binding domain, a transmembrane domain, a costimulatory signaling region, and a CD3 zeta signaling domain, and the antigen binding domain may bind to the marker.

In some embodiments, the predetermined condition may include the marker is present on the cell surface of a cell from that tumor cells are derived at least about at least one of 1.5-fold, 2-fold, 5-fold, 10-fold, 100-fold or 1000-fold greater than a suitable control cell, and the organ is non-essential such that an injury of the organ does not produce death of the subject.

In some embodiments, the non-essential organ is a mammary gland, and the marker is PRLR.

In some embodiments, the non-essential organ is a colon, and the marker is GUCY2C.

In some embodiments, the non-essential organ is a gastric gland, and the marker is Muc17.

In some embodiments, the non-essential organ is a bladder, and the marker is CD207.

In some embodiments, the non-essential organ is an ovary, and the marker is FZD10.

In some embodiments, the non-essential organ is a thyroid, and the marker is TSHR.

In some embodiments, the tumor is selected from a group consisting of breast cancer, a thyroid tumor, colorectal cancer, an ovary tumor, bladder cancer, and bladder cancer.

Some embodiments of the present disclosure relate to a modified cell including a nucleic acid sequence encoding a CAR having one of SEQ ID NOs: 36-42. For example, the cell is selected from the group consisting of a T cell, a natural killer (NK) cell, a cytotoxic T lymphocyte (CTL), and a regulatory T cell.

In some embodiments, the CAR has SEQ ID NO: 38, and an antigen binding domain of the CAR binds to prolactin receptor ligand having SEQ ID NO: 20 or 44.

In some embodiments, the CAR has SEQ ID NO: 40, and an antigen binding domain of the CAR binds to GUCY2C having SEQ ID NO: 33.

In some embodiments, the CAR has SEQ ID NO: 39, and an antigen binding domain of the CAR binds to Muc17 having SEQ ID NO: 31.

In some embodiments, the CAR has SEQ ID NO: 41, and an antigen binding domain of the CAR binds to CD207 having SEQ ID NO: 35.

In some embodiments, the CAR has SEQ ID NO: 36, and an antigen binding domain of the CAR binds to FZD10 having SEQ ID NO: 25.

In some embodiments, the CAR has SEQ ID NO: 37, and an antigen binding domain of the CAR binds to TSHR having SEQ ID NO: 27.

Some embodiments relate to a pharmaceutical composition comprising human T cells. The human T cells may include a nucleic acid sequence encoding a CAR; the CAR comprises an antigen binding domain, a transmembrane domain, a costimulatory signaling region, and a CD3 zeta signaling domain. The antigen binding domain may include one of amino acid sequences of SEQ ID NOs: 2, 5, 8, 11, 14, 17, or 20.

In some embodiments, the antigen binding domain comprises the amino acid sequence of SEQ ID NO: 2 and binds to Frizzled family receptor 10 (FZD10).

In some embodiments, the antigen binding domain comprises the amino acid sequence of SEQ ID NO: 5 and binds to Thyroid stimulating hormone receptor (TSHR).

In some embodiments, the CAR comprises the amino acid sequence of SEQ ID NO: 8 and binds to prolactin receptor (PRLR).

In some embodiments, the antigen binding domain comprises the amino acid sequence of SEQ ID NO: 11 and binds to Mucin 17 (Muc17).

In some embodiments, the antigen binding domain comprises the amino acid sequence of SEQ ID NO: 14 and binds to Guanylate cyclase 2C (GUCY2C).

In some embodiments, the antigen binding domain comprises the amino acid sequence of SEQ ID NO: 17 and binds to Langerin or Cluster of Differentiation 207 (CD207).

In some embodiments, the antigen binding domain comprises the amino acid sequence of SEQ ID NO: 20 and binds to PRLR.

In some embodiments, the CAR comprises the amino acid sequence of SEQ ID NO: 42 and binds to PRLR.

In some embodiments, the CAR comprises at least one of the amino acid sequences of SEQ ID NO: 36-42.

Some embodiments relate to a method for stimulating a T cell-mediated immune response to a cell population expressing an antigen. The method may include contacting the cell population with an effective amount of human T cells comprising a nucleic acid sequence encoding a CAR. The CAR may include an antigen binding domain, a transmembrane domain, a costimulatory signaling region, and a CD3 zeta signaling domain. The antigen binding domain may include one of amino acid sequences of SEQ ID NOs: 2, 5, 8, 11, 14, 17, or 20.

In some embodiments, the antigen binding domain comprises the amino acid sequence of SEQ ID NO: 2, and the antigen is FZD10.

In some embodiments, the antigen binding domain comprises the amino acid sequence of SEQ ID NO: 5, and the antigen is TSHR.

In some embodiments, the CAR comprises the amino acid sequence of SEQ ID NO: 8, and the antigen is PRLR.

In some embodiments, the antigen binding domain comprises the amino acid sequence of SEQ ID NO: 11, and the antigen is Muc17.

In some embodiments, the antigen binding domain comprises the amino acid sequence of SEQ ID NO: 14, and the antigen is GUCY2C.

In some embodiments, the antigen binding domain comprises the amino acid sequence of SEQ ID NO: 17, and the antigen is CD207.

In some embodiments, the antigen binding domain comprises the amino acid sequence of SEQ ID NO: 20, and the antigen is PRLR.

In some embodiments, the CAR comprises the amino acid sequence of SEQ ID NO: 42, and the antigen is PRLR.

In some embodiments, the CAR comprises at least one of the amino acid sequences of SEQ ID NO: 36-42.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

The Detailed Description is described with reference to the accompanying figures. The use of the same reference numbers in different figures indicates similar or identical items.

FIG. 1 illustrates multiple tumors and antigens of non-essential organs as well as corresponding genes thereof.

FIG. 2 shows the construction of antigen overexpressed K562 cell lines and includes images showing established target tumor cell lines.

FIG. 3 includes schematic diagrams illustrating the construction of CARS.

FIG. 4 shows assay results demonstrating that anti-CD207 CAR T cell lines were established.

FIG. 5 shows assay results demonstrating that anti-Muc17 CAR T cell lines were established.

FIG. 6 shows assay results demonstrating that anti-TSHR CAR T cell lines were established.

FIG. 7 shows assay results demonstrating that anti-PRLR CAR T cell lines were established.

FIG. 8 shows assay results demonstrating that anti-FZD10 CAR T cell lines were established.

FIG. 9 shows assay results demonstrating that anti-GUCY2C CAR T cell lines were established.

FIG. 10 shows co-cultivation assays demonstrating that anti-FZD10 CAR T cells recolonize specific tumor cells and release IFN-γ accordingly.

FIG. 11 shows co-cultivation assays demonstrating that anti-GUCY2C CAR T cells recolonize specific tumor cells and release IFN-γ accordingly.

FIG. 12 shows co-cultivation assays demonstrating that anti-Muc17 CAR T cells recolonize specific tumor cells and release IFN-γ accordingly.

FIG. 13 shows co-cultivation assays demonstrating that anti-CD207 CAR T cells recolonize specific tumor cells and release IFN-γ accordingly.

FIG. 14 shows co-cultivation assays demonstrating that anti-TSHR CAR T cells recolonize specific tumor cells and release IFN-γ accordingly.

FIG. 15 shows co-cultivation assays demonstrating that anti-PRLR CAR T cells and PRLR ligand CAR T cells recolonize specific tumor cells and release IFN-γ accordingly.

FIG. 16 shows killing assay results based on co-cultivation of anti-THSR CAR T cells and TSHR-K562 cells.

FIG. 17 shows killing assay results based on co-cultivation of anti-PRLR CAR T cells and PRLR-K562 cells.

FIG. 18 shows killing assay results based on co-cultivation of PRLR ligand CAR T cells and PRLR-K562 cells. Ligand 1 indicates human wild-type prolactin receptor ligand (SEQ ID NO: 20), and ligand 2 indicates modified human prolactin receptor ligand (SEQ ID NO: 44).

FIG. 19 is an image illustrating fluorescent signals observed from 3t3 cells expressing TSHR and fluorescent proteins (RFP).

FIG. 20 shows killing assay results based on co-cultivation of anti-TSHR CAR T cells and TSHR-3T3 cells.

FIG. 21 is an image illustrating fluorescent signals observed from 3t3 cells expressing PRLR and fluorescent proteins (RFP).

FIG. 22 shows killing assay results based on co-cultivation of anti-PRLR CAR T cells and PRLR-3T3 cells.

FIG. 23 shows killing assays results based on co-cultivation of PRLR ligand CAR T cells and PRLR-3T3 cells.

FIG. 24 is a series of images demonstrating in vivo antitumor activity of CAR T cells in accordance with embodiments of the present disclosure. Flow cytometry was used to evaluate the presence of K562-PRLR-RFP cells. In group A (upper images), two mice were injected with K562-PRLR-RFP cells, and buffer without human CAR T cells was transfused to these two mice. K562-PRLR-RFP cells (i.e., circled areas) were observed four weeks after injection. In group B (lower images), two mice were injected with K562-PRLR-RFP cells, and human CAR T cells were transfused to these two mice. K562-PRLR-RFP cells were not observed four weeks after injection (i.e., circled areas), showing the anti-tumor activity of the CAR T cells.

FIG. 25 is another series of images demonstrating in vivo antitumor activity of CAR T cells in accordance with embodiments of the present disclosure. Flow cytometry was used to evaluate the presence of CAR T cells in mice. Two mice were injected with K562-PRLR-RFP cells, and human CAR T cells were transfused to these two mice. Human CAR T cells (i.e., circled areas) were observed four weeks after injection, consistent with the anti-tumor activity of the CAR T cells shown in FIG. 24.

FIG. 26 shows in vivo antitumor activity of CAR T cells in established TNBC xenografts in immunodeficient mice. The upper diagram shows the establishment of the TNBC xenografts in immunodeficient mice and the measurement of antitumor activity. TNBC xenografts in immunodeficient mice were established by subcutaneously transplanting cells of MDA-MB-453 (TNBC) cell lines. After transplantation, NT/CAR T cells were transfused to the mice on Day 9, Day 15, and Day 30. Tumor volumes were observed and measured. The lower plot shows the in vivo antitumor activity of CAR T cells. The longitudinal axis represents tumor volumes, and the horizontal axis represents time after the transplantation.

### DETAILED DESCRIPTION

The present disclosure relates to compositions and methods for treating cancer among other diseases. The embodiments of the present disclosure include constructing a CAR including an antigen binding domain that binds to an antigen corresponding to a target gene. The target gene is specifically expressed in a certain tissue (e.g., a group of cells or an organ) or expressed in the tissue more than expressed in other tissues. In some embodiments, the CAR is expressed in modified cells (e.g., T-cells or NK cells), which are administrated to a subject having a tumor derived from cells of the tissue. Because the antigen is expressed in the tumor, the modified cells may identify and then cause these tumor cells to be killed. The modified cells are able to replicate and expand in vivo, and therefore are present long-term in the body of the subject, leading to sustained tumor control. In these instances, non-tumor cells that express the antigen may also be killed or damaged by the transferred CAR T/NK cells. However, since the tissue is selected from non-essential tissues with respect to the subject, the killing of normal cells of the tissue does not cause a life-threatening event (e.g., complications) to the subject. Examples of the nonessential tissues include organs such as prostate, breast, or melanocyte.

In some embodiments, antigens selected for CARS are mainly expressed in cancer cells and their primary organs (e.g., non-essential organs). In certain embodiments, a non-essential organ may be removed, before treating of CAR T cells, from a subject who has cancer in an advanced stage derived from the non-essential organ. For example, treatment for stage IV thyroid cancer is usually a combination of treatment techniques including surgery and radioactive iodine treatment. In these instances, the entire thyroid is removed, and the procedure is called a total thyroidectomy. Since selected antigens are mainly expressed in thyroid normal cells and thyroid cancer cells, the risk of CAR T cells attacking normal cells is reduced.

Generally, treatment of breast cancer includes a topical therapy and systemic therapy. Topical treatment includes mastectomy and radiotherapy, which bring patients great suffering and are only available for treating breast cancer in early stages. Systemic treatment includes chemotherapy, endocrine therapy, and targeted molecular therapies. Chemotherapy causes patients great pain, and its efficacy is poor. Endocrine therapy mainly applies to postmenopausal women. Molecular targeted therapy is one of the most active areas of research in recent years. However, its efficiency is relatively low. For example, recurrence rates of typical HER2 monoclonal antibody Herceptin monotherapy in the treatment of breast cancer are 15% to 30%.

Ovarian cancer is one of the common malignant tumors of female genital mutilation. Among ovarian cancer, epithelial cancer and malignant germ cell tumors are common. Most epithelial ovarian tumors will spread to the uterus, bilateral annex, omentum and pelvic organs, causing a serious threat to women's lives. Different pathological types of ovarian cancer have different treatment options. Therapies combined with surgery and chemotherapy may be used for treating ovarian cancer. For ovarian cancer in early stages, surgeries may be available and include comprehensive surgery and preserving fertility surgery. Because the complexity of ovarian embryonic development, tissue anatomy, and endocrine functions, symptoms for early stages are not typical; therefore, diagnosis of benign and malignant as well as tissue types is quite difficult. In the case of pelvic tumor metastasis, especially malignant germ cell tumors of patients in late stages, most of these patients cannot remove the lesion. In these instances, radiation and chemotherapy can only be used as adjunctive therapy, and their effect is limited.

Endometrial cancer occurs in a group of endometrial tumors, also called endometrial cancer. Tumors originated in endometrial glands are known as endometrial adenocarcinoma. These cancer cells mainly spread directly or via lymphatic metastasis, while for those in late stages, the cancer cells may spread to the lung, liver, and bone via blood metastasis. Treatment plans may be decided based on clinical stages, levels of tumor differentiation, and general conditions of patients. Generally, the treatment includes surgical treatment as well as radiotherapy, hormone, and chemotherapy. Conservative surgery may cause a relapse, while radical resection may cause symptoms of premature menopause. Radiation and chemotherapy can only be used as adjunctive therapies, and their effect is limited. Progestin therapy is effective using progesterone drugs to control the development of cancer, but the dosage is large, and long-term progestin therapies may impair liver functions. For patients with advanced tumor metastases, no effective treatment is available.

Cervical cancer is the most common form of female genital malignancies, including carcinoma in situ and invasive carcinoma. Cervical cancer is limited to the mucosa within the cortex, known as carcinoma in situ when no infiltration. When cancer stromal invasion is under mucous membranes, it is called invasive cancer. Cervical cancer can cause infertility in women. Since invasion and metastasis of advanced cancer, the symptoms may appear corresponding parts of the body as well. Generally, treatment plans are decided based on clinical stages, levels of tumor differentiation, and general conditions of patients. Generally, the treatment includes surgical treatment, as well as radiotherapy and chemotherapy as adjuvant therapy. Surgical treatment includes Radical hysterectomy and pelvic lymph node surgery Elimination, and there are recurrence risks. Radiation and chemotherapy can only be used as adjunctive therapies, and their effect is limited. For patients with advanced tumor metastases, currently, no effective treatment is available.

Thyroid cancer is the most common thyroid malignancy. There are four common thyroid cancer types: papillary (e.g., mixed papillary-follicular carcinoma), follicular, medullary (e.g., an entity with amyloidosis thyroid tumors) and undifferentiated carcinoma. Generally, treatment of papillary and follicular carcinoma is relatively effective, while medullary carcinoma is often transferred along lymphatic and blood roads and undifferentiated carcinoma is less common. Advanced thyroid cancer may produce hoarseness, breathing, difficulty swallowing, and Horner syndrome and sympathetic nerve compression caused by cervical plexus violations occur ear, pillows, shoulder pain, etc. and regional lymph nodes and distant metastasis, etc. There is a need for improving the level of prognosis and treatment of thyroid cancer. Current modes of treatment for thyroid cancer include surgery, postoperative radionuclide therapy, and endocrine therapy after surgery, wherein the surgical treatment is the first choice. In choices of surgery methods for treating differentiated thyroid cancer, many aspects of postoperative radioiodine treatment and TSH (thyroid stimulating hormone, TSH) suppression therapy, etc. have controversy. Also, there are no unified and standardized treatment guidelines, resulting in incomplete or excessive treatment. For advanced differentiated thyroid cancer, there is no effective treatment.

Colorectal cancer is a type of common gastrointestinal cancer, a serious threat to life and health. There is a need for improving the level of prognosis and treatment of colorectal cancer. Generally, clinical treatment programs for colorectal cancer include local excision, chemotherapy, and biologic therapy. The use of local excision of metastatic lesions: the standard of biological disease (example: synchronization and anisotropy) is important but difficult to be evaluated. For example, 75% of patients will relapse after liver metastases resection of the disease. Most of the relapsing occurs in livers, and its efficacy is limited. Commonly used chemotherapy regimens include FOLFOX, FOLFIRI, CapeOX, and FOLFOXIRI (2B), and other, However, combined with FOLFOX regimen 7%-8% of patients can extend 3-year PFS, while overall survival rates are not significant. Recent experimental data suggest that adding cetuximab to FOLFOX application for possible transfer patients with resectable lesions is harmful. Based on the current research data, combined with biological agents (e.g., bevacizumab, cetuximab, and panitumumab) treatment with these drugs have been recommended.

However, monoclonal antibody drugs generally have problems such as efficacy and persistence. Patients need long-term, repeated drug uses. At the same time, due to the combined treatment methods are generally stronger than monotherapy; therefore, whether other types of drugs will affect treatment and prognosis is still a question.

Stomach cancer (gastric cancer) is a malignant gastric epithelial origin, one of the most common gastrointestinal malignancies. Pain and weight loss are the most common clinical symptoms of advanced gastric cancer. Proliferation and metastasis pathway of gastric cancer cells include lymphatic metastasis, direct invasion, hematogenous metastasis, and peritoneal metastasis. There is a need for improving the level of prognosis and treatment of gastric cancer. Generally, gastric cancer treatment modes include surgery, chemotherapy, radiotherapy, immunotherapy, etc. But most cases of advanced gastric cancer have unresectable primary or metastatic lesions. Tumor metastasis and recurrence of gastric cancer are the leading causes of poor prognosis

Esophageal cancer (esophageal cancer, EC) is originated in the esophageal mucosal epithelial malignancies and is a common clinical malignant tumor. Tumor metastasis and recurrence of esophageal cancer are the leading causes of poor prognosis. Due to incidence and mortality, esophageal cancer is among the top ten in malignant tumors. There is a need for improving the level of prognosis and treatment of esophageal cancer. The current mode of treatment of esophageal cancer is a surgical combined therapy. There are problems in the staging of esophageal cancer, surgical treatment mode selection, surgical approach selection, lymphadenectomy way, and postoperative adjuvant therapy, etc.

Unless defined otherwise, all technical and scientific terms used herein have the same meaning as commonly understood by those of ordinary skill in the art to which the disclosure belongs. Although any methods and materials similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The term “activation,” as used herein, refers to the state of a T cell that has been sufficiently stimulated to induce detectable cellular proliferation. Activation can also be associated with induced cytokine production and detectable effector functions. The term “activated T cells” refers to, among other things, T cells that are undergoing cell division.

The term “antibody” is used in the broadest sense and specifically covers monoclonal antibodies (including full-length monoclonal antibodies), multispecific antibodies (e.g., bispecific antibodies), and antibody fragments so long as they exhibit the desired biological activity or function. The antibodies of the present disclosure may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

“Antibody fragments” comprise a portion of a full-length antibody, generally the antigen-binding or variable region of the antibody. Examples of antibody fragments include Fab, Fab′, F(ab′)2, and Fv fragments; diabodies; linear antibodies; single-chain antibody molecules; and multispecific antibodies formed from antibody fragments.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the Hand L chain) that contribute the amino acid residues for antigen binding and confer antigen binding specificity to the antibody. However, even a single variable domain (or half of a Fv including only three complementarity determining regions (CDRs) specific for an antigen) has the ability to recognize and bind antigen, although at a lower affinity than the entire binding site. An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. K and A light chains refer to the two major antibody light chain isotypes.

By the term, “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response, which may involve either antibody production, or the activation of specific immunologically competent cells, or both. Antigens may include any macromolecule, including virtually all proteins or peptides, or molecules derived from recombinant or genomic DNA. For example, DNA including a nucleotide sequence or a partial nucleotide sequence encoding a protein that elicits an immune response, therefore, encodes an “antigen” as that term is used herein. Furthermore, an antigen need not be encoded solely by a full-length nucleotide sequence of a gene. Further, an antigen can be generated, synthesized or derived from a biological sample including a tissue sample, a tumor sample, a cell or a biological fluid.

The term “anti-tumor effect” as used herein, refers to a biological effect associated with a decrease in tumor volume, a decrease in the number of tumor cells, a decrease in the number of metastases, an increase in life expectancy of a subject having tumor cells, or amelioration of various physiological symptoms associated with the cancerous condition. An “anti-tumor effect” can also be manifested by the ability of the peptides, polynucleotides, cells, and antibodies of the disclosure in the prevention of the occurrence of tumor in the first place.

The term “autoantigen” refers to an antigen mistakenly recognized by the immune system as being foreign. Auto-antigens include cellular proteins, phosphoproteins, cellular surface proteins, cellular lipids, nucleic acids, glycoproteins, including cell surface receptors.

The term “autologous” is used to describe a material derived from the same individual to which it is later to be re-introduced into the individual.

“Allogeneic” is used to describe a graft derived from a different animal of the same species.

“Xenogeneic” is used to describe a graft derived from an animal of a different species.

The term “cancer” as used herein is defined as a disease characterized by the rapid and uncontrolled growth of aberrant cells. Cancer cells can spread locally or through the bloodstream and lymphatic system to other parts of the body. Examples of various cancers include breast cancer, prostate cancer, ovarian cancer, cervical cancer, skin cancer, pancreatic cancer, colorectal cancer, renal cancer, liver cancer, brain cancer, lymphoma, leukemia, lung cancer et al.

Throughout this specification, unless the context requires otherwise, the words “comprise,” “includes” and “including” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands.

By “corresponds to” or “corresponding to” is meant (a) a polynucleotide having a nucleotide sequence that is substantially identical or complementary to all or a portion of a reference polynucleotide sequence or encoding an amino acid sequence identical to an amino acid sequence of a peptide or protein; or (b) a peptide or polypeptide having an amino acid sequence that is substantially identical to a sequence of amino acids in a reference peptide or protein.

“Co-stimulatory ligand,” includes a molecule on an antigen presenting cell (e.g., an APC, dendritic cell, B cell, et al.) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including proliferation, activation, differentiation, et al. A co-stimulatory ligand can include CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds to a costimulatory molecule present on a T cell, such as CD27, CD28, 4-1BB, OX40, CD30, CD40, PD-1, ICOS, lymphocyte function associated antigen-1 (LFA-1), CD2, CD7, LIGHT, NKG2C, B7-H3, and a ligand that specifically binds with CD83.

A “co-stimulatory molecule” refers to the cognate binding partner on a T cell that specifically binds to a costimulatory ligand, thereby mediating a co-stimulatory response by the T cell, such as proliferation. Co-stimulatory molecules include an MHC class I molecule, BTLA, and a Toll-like receptor.

A “co-stimulatory signal” refers to a signal, which in combination with a primary signal, such as TCR/CD3 ligation, leads to T cell proliferation and/or upregulation or downregulation of key molecules.

As used herein, the terms “disease” and “condition” may be used interchangeably or may be different in that the particular malady or condition may not have a known causative agent (so that etiology has not yet been worked out), and it is therefore not yet recognized as a disease but only as an undesirable condition or syndrome, wherein a more or less specific set of symptoms have been identified by clinicians. As used herein, a “disease” is a state of health of a subject wherein the subject cannot maintain homeostasis, and wherein if the disease is not ameliorated then the subject's health continues to deteriorate. In contrast, a “disorder” in a subject is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “effective” means adequate to accomplish a desired, expected, or intended result. For example, an “effective amount” may be an amount of a compound sufficient to produce a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

With regard to polynucleotides, the term “exogenous” refers to a polynucleotide sequence that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides, the term “endogenous” or “native” refers to naturally-occurring polynucleotide sequences that may be found in a given wild-type cell or organism. Also, a particular polynucleotide sequence that is isolated from a first organism and transferred to the second organism by molecular biological techniques is typically considered an “exogenous” polynucleotide with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The term “expression” as used herein is defined as the transcription and/or translation of a particular nucleotide sequence driven by its promoter.

“Expression vector” refers to a vector including a recombinant polynucleotide including expression control sequences operatively linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “immunoglobulin” or “Ig,” refers to a class of proteins, which function as antibodies. The five members included in this class of proteins are IgA, IgG, IgM, IgD, and IgE. IgA is the primary antibody that is present in body secretions, such as saliva, tears, breast milk, gastrointestinal secretions and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses, and is important in defense against bacteria and viruses. IgD is the immunoglobulin that has no known antibody function but may serve as an antigen receptor. IgE is the immunoglobulin that mediates immediate hypersensitivity by causing the release of mediators from mast cells and basophils upon exposure to the allergen.

By “isolated” is meant a material that is substantially or essentially free from components that normally accompany it in its native state. For example, an “isolated polynucleotide,” as used herein, refers to a polynucleotide, which has been purified from the sequences which flank it in a naturally-occurring state, e.g., a DNA fragment which has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment, and from association with other components of the cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

A “lentivirus” as used herein refers to a genus of the Reoviridae family. Lentiviruses are unique among the retroviruses in being able to infect nondividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

The nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “overexpressed” tumor antigen or “overexpression” of the tumor antigen is intended to indicate an abnormal level of expression of the tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), or intracisternal injection, or infusion techniques.

The terms “patient,” “subject,” “individual,” et al. are used interchangeably herein, and refer to any animal, or cells thereof whether in vitro or in situ, amenable to the methods described herein. In certain non-limiting embodiments, the patient, subject or individual is a human. In some embodiments, the term “subject” is intended to include living organisms in which an immune response can be elicited (e.g., mammals). Examples of subjects include humans, dogs, cats, mice, rats, and transgenic species thereof.

The recitation “polynucleotide” or “nucleic acid” as used herein designates mRNA, RNA, CRNA, rRNA, cDNA or DNA. The term typically refers to a polymeric form of nucleotides of at least 10 bases in length, either ribonucleotides or deoxynucleotides or a modified form of either type of nucleotide. The term includes single and double stranded forms of DNA and RNA.

The terms “polynucleotide variant” and “variant” and the like refer to polynucleotides displaying substantial sequence identity with a reference polynucleotide sequence or polynucleotides that hybridize to a reference sequence under stringent conditions that are defined hereinafter. These terms also encompass polynucleotides that are distinguished from a reference polynucleotide by the addition, deletion or substitution of at least one nucleotide. Accordingly, the terms “polynucleotide variant” and “variant” include polynucleotides in which one or more nucleotides have been added or deleted, or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs that encode these enzymes.

“Polypeptide,” “polypeptide fragment,” “peptide” and “protein” are used interchangeably herein to refer to a polymer of amino acid residues and to variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally occurring amino acid polymers. In certain aspects, polypeptides may include enzymatic polypeptides, or “enzymes,” which typically catalyze (i.e., increase the rate of) various chemical reactions.

The recitation polypeptide “variant” refers to polypeptides that are distinguished from a reference polypeptide sequence by the addition, deletion or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions and, in this regard, it is well understood in the art that some amino acids may be changed to others with broadly similar properties without changing the nature of the activity of the polypeptide. Polypeptide variants also encompass polypeptides in which one or more amino acids have been added or deleted, or replaced with different amino acid residues.

The term “promoter” as used herein is defined as a DNA sequence recognized by the synthetic machinery of the cell or introduced synthetic machinery, required to initiate the specific transcription of a polynucleotide sequence. The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

The term “bind,” “binds,” or “interacts with” means that one molecule recognizes and adheres to a particular second molecule in a sample or organism, but does not substantially recognize or adhere to other structurally unrelated molecules in the sample. By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross-reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

A “soluble receptor” is a receptor polypeptide that is not bound to a cell membrane. Soluble receptors are most commonly ligand-binding receptor polypeptides that lack transmembrane and cytoplasmic domains. Soluble receptors may include additional amino acid residues, such as affinity tags that provide for purification of the polypeptide or provide sites for attachment of the polypeptide to a substrate, or immunoglobulin constant region sequences. Many cell-surface receptors have naturally occurred, soluble counterparts that are produced by proteolysis. Soluble receptor polypeptides are said to be substantially free of transmembrane and intracellular polypeptide segments when they lack sufficient portions of these segments to provide membrane anchoring or signal transduction, respectively.

By “statistically significant,” it is meant that the result was unlikely to have occurred by chance. Statistical significance can be determined by any method known in the art. Commonly used measures of significance include the p-value, which is the frequency or probability with which the observed event would occur if the null hypothesis were true. If the obtained p-value is smaller than the significance level, then the null hypothesis is rejected. In simple cases, the significance level is defined at a p-value of 0.05 or less. A “decreased” or “reduced” or “lesser” amount is typically a “statistically significant” or a physiologically significant amount, and may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

By the term “stimulation,” is meant a primary response induced by binding of a stimulatory molecule (e.g., a TCR/CD3 complex) with its cognate ligand thereby mediating a signal transduction event, such as signal transduction via the TCR/CD3 complex. Stimulation can mediate altered expression of certain molecules, such as downregulation of TGF-β, and/or reorganization of cytoskeletal structures et al.

A “stimulatory molecule” refers to a molecule on a T cell that specifically binds to a cognate stimulatory ligand present on an antigen presenting cell.

A “stimulatory ligand” refers to a ligand that when present on an antigen presenting cell (e.g., an APC, a dendritic cell, a B-cell, et al.) can specifically bind with a cognate binding partner (referred to herein as a “stimulatory molecule”) on a T cell, thereby mediating a primary response by the T cell, including activation, initiation of an immune response, proliferation, et al. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to a cell that has been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “therapeutically effective amount” refers to the amount of the subject compound that will elicit the biological or medical response of tissue, system, or subject that is being sought by the researcher, veterinarian, medical doctor or another clinician. The term “therapeutically effective amount” includes that amount of a compound that, when administered, is sufficient to prevent the development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which includes an isolated nucleic acid and which can be used to deliver the isolated nucleic acid to the interior of a cell. Numerous vectors are known in the art including linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, et al. Examples of viral vectors include, but are not limited to, adenoviral vectors, adeno-associated virus vectors, retroviral vectors et al. For example, lentiviruses are complex retroviruses, which, in addition to the common retroviral genes gag, pol, and env, contain other genes with regulatory or structural function. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by attenuating the HIV virulence genes, for example, the genes env, vif, vpr, vpu, and nef are deleted making the vector biologically safe.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The present disclosure relates to isolated nucleic acid sequences, vectors including the isolated nucleic acid sequences, cells including the isolated nucleic acid sequences and methods of treating cancer using these cells.

Some embodiments of the present disclosure relate to an isolated nucleic acid sequence encoding a CAR. The CAR may include an antigen binding domain, a transmembrane domain, a costimulatory signaling region, and a CD3 zeta signaling domain. The antigen binding domain may bind to an antigen of a non-essential organ. For example, the antigen binding domain binds to an antigen that is expressed on the surface of a non-essential organ cell present in a microenvironment of a tumor.

As used herein, a non-essential organ refers to an organ of a subject, and the organ is non-essential such that an injury of the organ does not produce death of the subject. In some embodiments, an injury of the organ does not visibly affect the subject's health. For example, the prostate may be a non-essential organ for a male mammal, while breast may be a non-essential organ for a female mammal.

In certain embodiments, a non-essential organ may be removed, before treating of CAR T cells, from a subject who has cancer in an advanced stage derived from the non-essential organ. In these instances, the impact of treating of CAR T cells targeting antigens of the non-essential organ on the subject is substantially reduced. Examples of non-essential organs include a mammary gland, a colon, a gastric gland, an ovary, and a thyroid.

Some embodiments of the present disclosure relate to an isolated CAR including an antigen binding domain, a transmembrane domain, a costimulatory signaling region, and a CD3 zeta signaling domain. The antigen binding domain may bind to an antigen of a non-essential organ.

Some embodiments of the present disclosure relate to a pharmaceutical composition including human T cells. The human T cells may include a nucleic acid sequence encoding a CAR. The CAR may include an antigen binding domain, a transmembrane domain, a costimulatory signaling region, and a CD3 zeta signaling domain. The antigen binding domain may bind to an antigen of a non-essential organ.

Some embodiments of the present disclosure relate to a cell including a nucleic acid sequence encoding a CAR. The CAR may include an antigen binding domain, a transmembrane domain, a costimulatory signaling region, and a CD3 zeta signaling domain. The antigen binding domain binds to an antigen of a non-essential organ. For example, the cell is selected from the group consisting of a T cell, a natural killer (NK) cell, NK-92 cell, a cytotoxic T lymphocyte (CTL), and a regulatory T cell.

Some embodiments of the present disclosure relate to a vector comprising a nucleic acid sequence encoding a CAR. The CAR may include an antigen binding domain, a transmembrane domain, a costimulatory signaling region, and a CD3 zeta signaling domain. The antigen binding domain may bind to an antigen of a non-essential organ.

Some embodiments of the present disclosure relate to a method for stimulating a T cell-mediated immune response to a cell population in a non-essential organ of a subject. The method may include administering to a subject an effective amount of a cell genetically modified to express a CAR. The CAR may include an antigen binding domain, a transmembrane domain, a costimulatory signaling region, and a CD3 zeta signaling domain. The antigen binding domain is selected to recognize the cell population of the non-essential organ specifically.

In some embodiments, the T cells may be modified to have a disruption in an endogenous gene associated with a biosynthesis or transportation pathway of one or more proteins. Examples of the one or more proteins include Programmed cell death protein 1 (PD-1) gene and Major Histocompatibility Complex I (MHC I). For example, Major Histocompatibility Complex of T cells may be disrupted by modification of Beta-2-microglobulin (B2M) gene, antigen presentation 1 (TAP1) gene, and TAP-associated glycoprotein (TAPBP) gene in the T cells. In certain embodiments, the disruption may be introduced into a T cell before or after the T cell is transferred with a nucleic acid sequence encoding a CAR.

Some embodiments of the present disclosure relate to a method of treating a subject with cancer. The method may include administering to the subject a cell genetically engineered to express a CAR. The CAR may include an antigen binding domain, a transmembrane domain, a costimulatory signaling region, and a CD3 zeta signaling domain. The antigen binding domain may bind to an antigen of a non-essential organ. For example, the cell is selected from the group consisting of a T cell, a natural killer (NK) cell, NK-92 cell, a cytotoxic T lymphocyte (CTL), and a regulatory T cell.

CARs are molecules generally including an extracellular and intracellular domain. The extracellular domain includes a target-specific binding element. The intracellular domain (e.g., cytoplasmic domain) includes a costimulatory signaling region and a zeta chain portion. The costimulatory signaling region refers to a portion of the CAR including the intracellular domain of a costimulatory molecule. Costimulatory molecules are cell surface molecules other than antigens receptors or their ligands that are required for an efficient response of lymphocytes to antigen.

Between the extracellular domain and the transmembrane domain of the CAR, there may be incorporated a spacer domain. As used herein, the term “spacer domain” generally means any oligo- or polypeptide that functions to link the transmembrane domain to, either the extracellular domain or, the cytoplasmic domain of the polypeptide chain. A spacer domain may include up to 300 amino acids, preferably 10 to 100 amino acids, and most preferably 25 to 50 amino acids.

In some embodiments, the target-specific binding element of the CAR in the present disclosure may recognize a tumor antigen. Tumor antigens are proteins that are produced by tumor cells that elicit an immune response, particularly T-cell mediated immune responses. Tumor antigens are well known in the art and include, for example, a glioma-associated antigen, carcinoembryonic antigen (CEA), β-human chorionic gonadotropin, alpha-fetoprotein (AFP), lectin-reactive AFP, thyroglobulin, RAGE-1, MN-CA IX, human telomerase reverse transcriptase, RU1, RU2 (AS), intestinal carboxylesterase, mut hsp70-2, M-CSF, prostase, prostate-specific antigen (PSA), PAP, NY-ESO-1, LAGE-1a, p53, prostein, PSMA, Her2/neu, survivin and telomerase, prostate carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

In some embodiments, the tumor antigen includes HER2, CD19, CD20, CD22, Kappa or light chain, CD30, CD33, CD123, CD38, ROR1, ErbB3/4, EGFR, EGFRvIII, EphA2, FAP, carcinoembryonic antigen, EGP2, EGP40, mesothelin, TAG72, PSMA, NKG2D ligands, B7-H6, IL-13 receptor α 2, IL-11 receptor α, MUC1, MUC16, CA9, GD2, GD3, HMW-MAA, CD171, Lewis Y, G250/CAIX, HLA-AI MAGE A1, HLA-A2 NY-ESO-1, PSC1, folate receptor-α, CD44v7/8, 8H9, NCAM, VEGF receptors, 5T4, Fetal AchR, NKG2D ligands, CD44v6, TEM1, TEM8, or viral-associated antigens expressed by the tumor.

In some embodiments, the binding element of the CAR may include any antigen binding moiety that when bound to its cognate antigen, affects a tumor cell such that the tumor cell fails to grow, or is promoted to die or diminish.

In some embodiments, the antigen binding element of the CAR of the disclosure targets an antigen of a non-essential organ. In some instances, the antigen binding element of the CAR of the disclosure includes anti-antigen including the amino acid sequence set forth at least at one of SEQ ID NOs: 2-20 or 44.

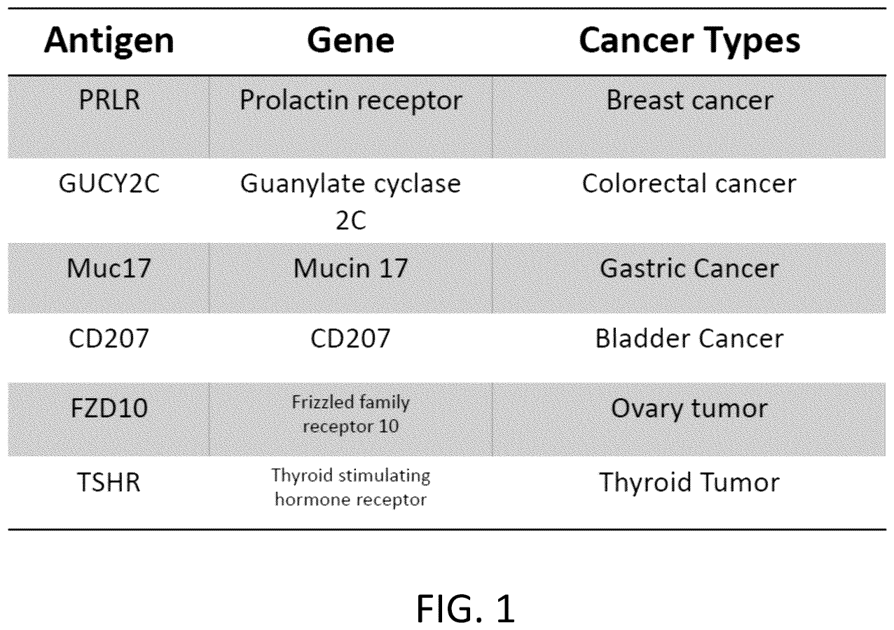

In some embodiments, internal ribosome entry sites (IRES) elements are used to create multigene, or polycistronic, or messages. For example, an IRES element may link a nucleic acid sequence encoding CAR and a nucleic acid sequence encoding one of the various antigens (See FIG. 1 and Table 2). In other embodiments, other tools such as 2A may be used to create multigene, or polycistronic, or messages.

The nucleic acid sequences coding for the desired molecules can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

The embodiments of the present disclosure further relate to vectors in which a DNA of the present disclosure is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from oncoretroviruses such as murine leukemia viruses in that they can transduce non-proliferating cells, such as hepatocytes. They also have the added advantage of low immunogenicity.

The expression of natural or synthetic nucleic acids encoding CARs is typically achieved by operably linking a nucleic acid encoding the CAR polypeptide or portions thereof to one or more promoters and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

In some embodiments, the antigen binding domain is an antibody, an antigen-binding fragment thereof, or a ligand thereof. For example, the antigen-binding fragment is a Fab or a scFv.

In some embodiments, the antigen is expressed on a non-essential organ cell present in a microenvironment of a tumor.

In some embodiments, the tumor is a breast cancer. In certain embodiments, the antigen is a mammary gland antigen. For example, the mammary gland antigen is prolactin receptor (PRLR) having SEQ ID NO: 29.

As used herein, “a mammary gland antigen” refers to an antigen expressed on or by a mammary gland cell. Examples of mammary gland cells include catheter epithelial cells/foam cells.

The prolactin receptor (PRLR) is involved in the growth and differentiation of various cells. Prolactin receptors have been identified in a number of cells and tissues, including the mammary gland, organs of the reproductive system, central nervous system, pituitary, adrenal cortex, skin, bone, lung, heart, liver, pancreas, GI tract, kidney and lymphoid tissues. Human growth hormone (hGH), human prolactin (hPRL) and human placental lactogen (hPL) all specifically bind the prolactin receptor with high affinity.

As used herein, “PRLR” refers to human prolactin receptor. The term should be construed to include not only human prolactin receptor but variants, homologs, fragments and portions thereof to the extent that such variants, homologs, fragments and portions thereof retain the ability of prolactin receptor to bind to antibodies or ligands of human prolactin receptor as disclosed herein.

In some embodiments, the nucleotide sequence encoding at least a portion of the human prolactin receptor is shown in SEQ ID NO: 28 and at least a portion of the human prolactin receptor are shown in SEQ ID NO: 29.

In some embodiments, the present disclosure is particularly well-suited to deliver agents to cells that overexpress the prolactin receptor differentially. As used herein, a prolactin receptor is “overexpressed” when it is present on the surface of a cell (e.g., a mammary gland cell) in an amount that is statistically significantly greater than a suitable control cell (e.g., a brain cell). In some embodiments, the prolactin receptor is present on the cell surface at least about 1.5-fold, 2-fold, 5-fold, 10-fold, 100-fold or 1000-fold greater than a suitable control cell. For example, over 80% of breast tumors overexpress the prolactin receptor by as much as 10- to 1000-fold over normal breast tissue. Accordingly, some embodiments of the present disclosure are particularly suitable for delivery of an agent (e.g., CAR T/NK cells) to breast cancer cells of a subject. In these instances, non-tumor cells that express the prolactin receptor may also be killed by the transferred CAR T/NK cells. However, if the mammary gland is non-essential tissues with respect to the subject, the killing of normal cells of the mammary gland does not cause a life-threatening event (e.g., complications) to the subject.

In some embodiments, the antigen binding domain is prolactin receptor ligand having SEQ ID NO: 20 or 44.

A prolactin receptor ligand, as used herein, refers to an entity that binds the prolactin receptor, regardless of whether downstream biological effects of prolactin receptor binding are observed. As will be appreciated, no particular level of binding specificity is required, and acceptable levels of specificity will depend on the application. Suitable prolactin receptor ligands include human placental lactogen and variants thereof (including truncated or modified forms), prolactin and variants thereof (including truncated or modified forms) and human growth hormone (hGH) (including truncated or modified forms). Selection of a suitable prolactin receptor ligand will depend on, e.g., the agent to be delivered, the coupling strategy to be used, and the degree of receptor activation desired, if any.

In some embodiments, at least a portion of prolactin receptor ligand is shown in SEQ ID NO: 20 or 44.

In some embodiments, the tumor is colorectal cancer. In certain embodiments, the antigen is a colon antigen. For example, the colon antigen is Guanylate cyclase 2C (GUCY2C) having SEQ ID NO: 33.

As used herein, “a colon antigen” refers to an antigen expressed on or by a colon cell. Examples of colon cells include goblet cells and enterocytes.

Guanylyl cyclase 2C (GUCY2C) is principally expressed in intestinal epithelial cells. GUCY2C is the receptor for diarrheagenic bacterial enterotoxins (STs) and the gut paracrine hormones, guanylin, and uroguanylin. These ligands regulate water and electrolyte transport in the intestinal and renal epithelia and are ultimately responsible for acute secretory diarrhea.

As used herein, “GUCY2C” refers to human Guanylyl cyclase 2C. The term should be construed to include not only human Guanylyl cyclase 2C, but variants, homologs, fragments and portions thereof to the extent that such variants, homologs, fragments and portions thereof retain the ability of Guanylyl cyclase 2C to bind to antibodies or ligands of human Guanylyl cyclase 2C as disclosed herein.

In some embodiments, the nucleotide sequence encoding at least a portion of GUCY2C is shown in SEQ ID NO: 32 and the amino acid sequence of at least a portion of GUCY2C are shown in SEQ ID NO: 33.

In some embodiments, the present disclosure is particularly well-suited to deliver agents to cells that overexpress the GUCY2C differentially. As used herein, a GUCY2C is “overexpressed” when it is present on the surface of a cell (e.g., a colon cell) in an amount that is statistically significantly greater than a suitable control cell (e.g., a brain cell and a pancreas cell). In some embodiments, the GUCY2C is present on the cell surface at least about 1.5-fold, 2-fold, 5-fold, 10-fold, 100-fold or 1000-fold greater than a suitable control cell. Accordingly, some embodiments of the present disclosure are particularly suitable for delivery of an agent (e.g., CAR T/NK cells) to colorectal cancer cells. In these instances, non-tumor cells that express the GUCY2C may also be killed by the transferred CAR T/NK cells. However, if the colon is non-essential tissues with respect to the subject, the killing of normal cells of the colon does not cause a life-threatening event (e.g., complications) to the subject.

In some embodiments, the tumor is gastric cancer. In certain embodiments, the antigen is a gastric gland antigen. For example, the gastric gland antigen is Mucin 17 (Muc17) having SEQ ID NO: 31.

As used herein, “a gastric gland antigen” refers to an antigen expressed on or by a gastric gland cell. Examples of gastric gland cells include gastric parietal cells, mucous cells, and surface epithelial cells.

Muc17 is a type 1 membrane protein comprising 4,493 amino acids. Muc17 belongs to the membrane-form mucin family, and most part of its extracellular domain comprises a tandem repeat of a serine-, threonine-, and proline-rich 59-mer sequence and is glycosylated.

As used herein, “Muc17” refers to human Mucin 17. The term should be construed to include not only human Mucin 17 but variants, homologs, fragments and portions thereof to the extent that such variants, homologs, fragments and portions thereof retain the ability of Mucin 17 to bind to antibodies or ligands of human Mucin 17 as disclosed herein.

In some embodiments, the nucleotide sequence encoding at least a portion of Muc17 is shown in SEQ ID NO: 30 and the amino acid sequence of at least a portion of Muc17 are shown in SEQ ID NO: 31.

In some embodiments, the present disclosure is particularly well-suited to deliver agents to cells that overexpress the Muc17 differentially. As used herein, a Muc17 is “overexpressed” when it is present on the surface of a cell (e.g., a gastric gland cell) in an amount that is statistically significantly greater than a suitable control cell (e.g., a brain cell and a pancreas cell). In some embodiments, the Muc17 is present on the cell surface at least about 1.5-fold, 2-fold, 5-fold, 10-fold, 100-fold or 1000-fold greater than a suitable control cell. Accordingly, some embodiments of the present disclosure are particularly suitable for delivery of an agent to gastric cancer cells. In these instances, non-tumor cells that express the Muc17 may also be killed by the transferred CAR T/NK cells. However, if the gastric gland is non-essential tissues with respect to the subject, the killing of normal cells of the colon does not cause a life-threatening event (e.g., complications) to the subject.

In some embodiments, the tumor is a bladder cancer. In certain embodiments, the antigen is a bladder antigen. For example, the bladder antigen is CD207 having SEQ ID NO: 35.

As used herein, “a bladder antigen” refers to an antigen expressed on or by a bladder cell. Examples of bladder cells include transitional cells and mucosal epithelial cells.

CD207 (langerin or Cluster of Differentiation 207) is a protein which in humans is encoded by the CD207 gene. CD207 is a type II transmembrane, C-type lectin receptor on Langerhans cells. CD207 is localized in the Birbeck granules, organelles present in the cytoplasm of Langerhans cells and including superimposed and zippered membranes.

As used herein, “CD207” refers to human CD207. The term should be construed to include not only human CD207 but variants, homologs, fragments and portions thereof to the extent that such variants, homologs, fragments and portions thereof retain the ability of human CD207 to bind to antibodies or ligands of human CD207 as disclosed herein.

In some embodiments, the nucleotide sequence encoding at least a portion of the human CD207 is shown in SEQ ID NO: 34 and the amino acid sequence of at least a portion of the human CD207 are shown in SEQ ID NO: 35.

In some embodiments, the present disclosure is particularly well-suited to deliver agents to cells that overexpress the CD207 differentially. As used herein, a CD207 is “overexpressed” when it is present on the surface of a cell (e.g., a bladder cell) in an amount that is statistically significantly greater than a suitable control cell (e.g., an endocrine tissue cell). In some embodiments, the CD207 is present on the cell surface at least about 1.5-fold, 2-fold, 5-fold, 10-fold, 100-fold or 1000-fold greater than a suitable control cell. Accordingly, some embodiments of the present disclosure are particularly suitable for delivery of an agent to bladder cancer cells. In these instances, non-tumor cells that express the CD207 may also be killed by the transferred CAR T/NK cells. However, if the bladder is non-essential tissues with respect to the subject, the killing of normal cells of the colon does not cause a life-threatening event (e.g., complications) to the subject.

In some embodiments, the tumor is an ovary tumor. In certain embodiments, the antigen is an ovary antigen. For example, the ovary antigen is Frizzled family receptor 10 (FZD10) having SEQ ID NO: 25.

As used herein, “an ovary antigen” refers to an antigen expressed on or by an ovary cell. Examples of ovary cells include follicular cells, granulosa cells, and germinal epithelium.

A human Fz gene family member, Frizzled-10 (FZD10), has been cloned and characterized. Analysis of the FZD10 nucleotide sequence showed that the human FZD10 gene encodes a seven-transmembrane-receptor of 581 amino acids, including an amino-terminal cysteine-rich domain and a carboxy-terminal Ser/Thr-Xxx-Val motif. FZD10-encoding mRNA (4.0 kb) was detected in placenta, fetal kidney, fetal lung, and brain. In adult brain, FZD10 mRNA was abundant in the cerebellum. The FZD10 gene was mapped to human chromosome 12q24.33. FZD10 shares 65.7% amino-acid identity with Frizzled-9 (FZD9). FZD10 and FZD9 constitute a subfamily of the Frizzled genes. FZD10 is the receptor for the Wnt ligand proteins WNT7a and WNT7b. There is 93% identity between mouse and human FZD10.

As used herein, “FZD10” refers to human FZD10. The term should be construed to include not only human FZD10 but variants, homologs, fragments and portions thereof to the extent that such variants, homologs, fragments and portions thereof retain the ability of human FZD10 to bind to antibodies or ligands of human FZD10 as disclosed herein.

In some embodiments, the nucleotide sequence encoding at least a portion of the human FZD10 is shown in SEQ ID NO: 24 and the amino acid sequence of at least a portion of the human FZD10 are shown in SEQ ID NO: 25.

In some embodiments, the present disclosure is particularly well-suited to deliver agents to cells that overexpress the FZD10 differentially. As used herein, an FZD10 is “overexpressed” when it is present on the surface of a cell (e.g., an ovary cell) in an amount that is statistically significantly greater than a suitable control cell (e.g., an endocrine tissue cell). In some embodiments, the FZD10 is present on the cell surface at least about 1.5-fold, 2-fold, 5-fold, 10-fold, 100-fold or 1000-fold greater than a suitable control cell. Accordingly, some embodiments of the present disclosure are particularly suitable for delivery of an agent to ovary cancer cells. In these instances, non-tumor cells that express the FZD10 may also be killed by the transferred CAR T/NK cells. However, if the ovary is non-essential tissues with respect to the subject, the killing of normal cells of the colon does not cause a life-threatening event (e.g., complications) to the subject.

In some embodiments, the tumor is a thyroid tumor. In certain embodiments, the antigen is a thyroid antigen. For example, the thyroid antigen is Thyroid stimulating hormone receptor (TSHR) having SEQ ID NO: 27.

As used herein, “a thyroid antigen” refers to an antigen expressed on or by a thyroid cell. Examples of thyroid cells include follicular cells and parafollicular cells.

A human TSHR is a receptor for thyroid-stimulating hormone (TSH) which is present on the thyroid membrane. When TSH secreted from the pituitary gland binds to TSHR on the thyroid follicle cell membrane, the thyroid gland secretes T3 and T4 having metabolic functions. TSHR is a seven-transmembrane receptor having a molecular weight of about 95,000 to 100,000. It was reported that the human thyrotropin receptor (TSHR) includes three domains: a leucine-rich domain (LRD; amino acids 36-281), a cleavage domain (CD; amino acids 282-409), and transmembrane domain (TMD; amino acids 410-699). Human thyrotropin (hTSH) a chains were found to make contact with many amino acids on the LRD surface and CD surface.

As used herein, “TSHR” refers to human thyroid stimulating hormone receptor. The term should be construed to include not only human thyroid stimulating hormone receptor, but variants, homologs, fragments and portions thereof to the extent that such variants, homologs, fragments and portions thereof retain the ability of human thyroid stimulating hormone receptor to bind to antibodies or ligands of human thyroid stimulating hormone receptor as disclosed herein.

In some embodiments, the nucleotide sequence encoding at least a portion of the human thyroid stimulating hormone receptor is shown in SEQ ID NO: 26 and the amino acid sequence of at least a portion of the human thyroid stimulating hormone receptor are shown in SEQ ID NO: 27.

In some embodiments, the present disclosure is particularly well-suited to deliver agents to cells that overexpress the TSHR differentially. As used herein, a TSHR is “overexpressed” when it is present on the surface of a cell (e.g., a thyroid cell) in an amount that is statistically significantly greater than a suitable control cell (e.g., a brain cell or a pancreas cell). In some embodiments, the TSHR is present on the cell surface at least about 1.5-fold, 2-fold, 5-fold, 10-fold, 100-fold or 1000-fold greater than a suitable control cell. Accordingly, some embodiments of the present disclosure are particularly suitable for delivery of an agent to thyroid cancer cells. In these instances, non-tumor cells that express the TSHR may also be killed by the transferred CAR T/NK cells. However, if the thyroid is non-essential tissues with respect to the subject, the killing of normal cells of the colon does not cause a life-threatening event (e.g., complications) to the subject.

In some embodiments, the costimulatory signaling region may include the intracellular domain of a costimulatory molecule selected from the group consisting of CD27, CD28, 41-BB, OX40, CD30, CD40, PD-1, ICOS, lymphocyte function associated antigen-1 (LFA-1), CD2, CD7, LIGHT, NKG2C, B7-H3, a ligand that specifically binds with CD83, and any combination thereof.

In some embodiments, the antigen binding domain may include at least one of SEQ ID NOs: 2-20 or 44.

In some embodiments, the antigen binding domain may include SEQ ID NO: 3 or 4, or a combination thereof, and the tumor is ovary tumor.

In some embodiments, the antigen binding domain may include SEQ ID NO: 6 or 7, or a combination thereof, and the tumor is thyroid tumor.

In some embodiments, the antigen binding domain may include SEQ ID NO: 9 or 10, or a combination thereof, and the tumor is breast cancer.

In some embodiments, the antigen binding domain may include SEQ ID NO: 12 or 13, or a combination thereof, and the tumor is gastric cancer.

In some embodiments, the antigen binding domain may include SEQ ID NO: 15 or 16, or a combination thereof, and the tumor is colorectal cancer.

In some embodiments, the antigen binding domain may include SEQ ID NO: 18 or 19, or a combination thereof, and the tumor is bladder cancer.

In some embodiments, the antigen binding domain may include SEQ ID NO: 20 or 44, and the tumor is breast cancer.

Some embodiments of the present disclosure relate to a method of selecting an antigen binding domain for a CAR for treating a subject with tumor cells. The method may include determining an organ of cells from that the tumor cells derived, determining that the organ is a non-essential organ with respect to the subject, searching a database to identify multiple markers that are expressed in a cell population of the organ, selecting a marker of the multiple markers based on a predetermined condition, and generating cells comprising a CAR using cells from the subject. The CAR may include an antigen binding domain, a transmembrane domain, a costimulatory signaling region, and a CD3 zeta signaling domain, and the antigen binding domain may bind to the marker. Examples of an organ include mammary gland, gastric gland, etc. In certain embodiments, the organ does not include a blood tissue.

In some embodiments, the predetermined condition may include the marker is present on the cell surface of a cell from that tumor cells are derived at least about at least one of 1.5-fold, 2-fold, 5-fold, 10-fold, 100-fold or 1000-fold greater than a suitable control cell, and the organ is non-essential such that an injury of the organ does not produce death of the subject.

In some embodiments, the non-essential organ is a mammary gland, and the marker is PRLR.

In some embodiments, the non-essential organ is a colon, and the marker is GUCY2C.

In some embodiments, the non-essential organ is a gastric gland, and the marker is Muc17.

In some embodiments, the non-essential organ is a bladder, and the marker is CD207.

In some embodiments, the non-essential organ is an ovary, and the marker is FZD10.

In some embodiments, the non-essential organ is a thyroid, and the marker is TSHR.

In some embodiments, the tumor is selected from a group consisting of breast cancer, a thyroid tumor, colorectal cancer, an ovary tumor, bladder cancer, and is bladder cancer.

Some embodiments of the present disclosure relate to a modified cell including a nucleic acid sequence encoding a CAR having one of SEQ ID NOs: 36-42. For example, the cell is selected from the group consisting of a T cell, a natural killer (NK) cell, a cytotoxic T lymphocyte (CTL), and a regulatory T cell.

In some embodiments, the CAR has SEQ ID NO: 38, and an antigen binding domain of the CAR binds to prolactin receptor ligand having SEQ ID NO: 20 or 44.

In some embodiments, the CAR has SEQ ID NO: 40, and an antigen binding domain of the CAR binds to GUCY2C having SEQ ID NO: 33.

In some embodiments, the CAR has SEQ ID NO: 39, and an antigen binding domain of the CAR binds to Muc17 having SEQ ID NO: 31.

In some embodiments, the CAR has SEQ ID NO: 41, and an antigen binding domain of the CAR binds to CD207 having SEQ ID NO: 35.

In some embodiments, the CAR has SEQ ID NO: 36, and an antigen binding domain of the CAR binds to FZD10 having SEQ ID NO: 25.

In some embodiments, the CAR has SEQ ID NO: 37, and an antigen binding domain of the CAR binds to TSHR having SEQ ID NO: 27.

Additional information related to expression synthetic nucleic acids encoding CARs and gene transfer into mammalian cells is provided in U.S. Pat. No. 8,906,682, incorporated by reference in its entirety.

The embodiments further relate to methods for treating a patient for an illness including administering to the patient an effective amount of the engineered cells of the present disclosure. Various illnesses can be treated according to the present methods including cancer, such as ovarian carcinoma, breast carcinoma, colon carcinoma, glioblastoma multiforme, prostate carcinoma and leukemia. In some embodiments, the method includes administering to a human patient a pharmaceutical composition including an effective antitumor amount of a population of human T cells, wherein the human T cells of the population include human T-cells that comprise the nucleic acid sequence as described in the present disclosure.

Cancers that may be treated include tumors that are not vascularized, or not yet substantially vascularized, as well as vascularized tumors. The cancers may include non-solid tumors (such as hematological tumors, for example, leukemias and lymphomas) or may include solid tumors. Types of cancers to be treated with the CARS of the disclosure include, but are not limited to, carcinoma, blastoma, and sarcoma, and certain leukemia or lymphoid malignancies, benign and malignant tumors, and malignancies, e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brain stem glioma and mixed gliomas), glioblastoma (also known as glioblastoma multiforme) astrocytoma, CNS lymphoma, germinoma, medulloblastoma, Schwannoma craniopharyngioma, ependymoma, pinealoma, hemangioblastoma, acoustic neuroma, oligodendroglioma, meningioma, neuroblastoma, retinoblastoma and brain metastases).

Generally, the cells activated and expanded as described herein may be utilized in the treatment and prevention of diseases that arise in individuals who are immunocompromised. In particular, the engineered cells of the present disclosure are used in the treatment of cancer. In certain embodiments, the cells of the present disclosure are used in the treatment of patients at risk of developing cancer. Thus, the present disclosure provides methods for the treatment or prevention of cancer comprising administering to a subject in need thereof, a therapeutically effective amount of the engineered T cells of the present disclosure.

The engineered T cells of the present disclosure may be administered either alone or as a pharmaceutical composition in combination with diluents and/or with other components such as IL-2 or other cytokines or cell populations. Briefly, pharmaceutical compositions of the present disclosure may include a target cell population as described herein, in combination with one or more pharmaceutically or physiologically acceptable carriers, diluents or excipients. Such compositions may include buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure are preferably formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

When “an immunologically effective amount”, “an anti-tumor effective amount”, “a tumor-inhibiting effective amount”, or “therapeutic amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, preferably 10⁵ to 10₆ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regimen for a particular patient can readily be determined by one skilled in the art of medicine by monitoring the patient for signs of disease and adjusting the treatment accordingly.

In certain embodiments, it may be desired to administer activated T cells to a subject and then subsequently redraw blood (or have an apheresis performed), activate T cells therefrom according to the present disclosure, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocols, may select out certain populations of T cells.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodal, intramedullary, intramuscularly, by intravenous (i. v.) injection, or intraperitoneally. In one embodiment, the T cell compositions of the present disclosure are administered to a patient by intradermal or subcutaneous injection. In another embodiment, the T cell compositions of the present disclosure are preferably administered by i.v. injection. The compositions of T cells may be injected directly into a tumor, lymph node, or site of infection.

In certain embodiments of the present disclosure, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells of the present disclosure may be used in combination with chemotherapy, radiation, immunosuppressive agents, such as cyclosporin, azathioprine, methotrexate, mycophenolate, and FK506, antibodies, or other immunoablation agents such as CAM PATH, anti-CD3 antibodies or other antibody therapies, Cytoxan, fludarabine, cyclosporine, FK506, rapamycin, mycophenolic acid, steroids, FR901228, cytokines, and irradiation. These drugs inhibit either the calcium-dependent phosphatase calcineurin (cyclosporine and FK506) or inhibit the p70S6 kinase that is important for growth factor-induced signaling (rapamycin). (Liu et al., Cell 66:807-815, 1991; Henderson et al., Immun 73:316-321, 1991; Bierer et al., Curr. Opin. Immun 5:763-773, 1993; Isoniemi (supra)). In a further embodiment, the cell compositions of the present disclosure are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present disclosure are administered following B-cell ablative therapy such as agents that react with CD20, e.g., Rituxan. For example, in one embodiment, subjects may undergo standard treatment with high-dose chemotherapy followed by peripheral blood stem cell transplantation. In certain embodiments, following the transplant, subjects receive an infusion of the expanded immune cells of the present disclosure. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period of 1 and 30 days. The preferred daily dose is 1 to 10 mg per day although in some instances larger doses of up to 40 mg per day may be used (described in U.S. Pat. No. 6,120,766, incorporated by reference in its entirety).

Additional information on the methods of cancer treatment using engineer T cells is provided in U.S. Pat. No. 8,906,682, incorporated by reference in its entirety.

Some embodiments of the present disclosure relate to a nucleic acid sequence encoding a CAR. For example, the CAR includes an antigen binding domain, a transmembrane domain, and an intracellular domain. The antigen binding domain binds to an organ lineage antigen, and the organ lineage antigen is expressed in a tumor and normal tissue from which the tumor is derived.

The terms “tumor associated antigens” as used herein refer to antigens selectively expressed or overexpressed by malignant cells as compared with normal adult tissue. The tumor associated antigens include various groups such as tumor specific antigens, oncofetal antigens, oncogene products, organ lineage antigens, viral antigens, etc. For example, oncogene and suppressor gene products, such as nonmutated HER-2/neu and p53, are analogous to oncofetal antigens in that they can be overexpressed in tumors and may be expressed in some fetal tissues.

The term “tumor specific antigens” as used herein refers to antigens that are uniquely expressed in tumors, such as point-mutated ras oncogenes, p53 mutations, anti-idiotype antibodies (Abs), and products of ribonucleic acid (RNA) splice variants and gene translocations.

The term “organ lineage antigen” as used herein is defined an antigen expressed in a tumor of a given type and the normal organ from which the tumor is derived. Examples of organ lineage antigen include prostate-specific antigen (PSA) and the melanocyte antigens, such as MART-1/Melan A, tyrosinase, gp100, and TRP-1/gp75. Organ lineage antigens may be targets for immunotherapy if the normal organ in which they are expressed is not essential, such as the prostate, breast, or melanocyte. As used herein, an organ refers to an integrated group of cells with a common structure, an intercellular material, and/or a function.

Some embodiments of the present disclosure relate to a vector including the nucleic acid sequence. In some embodiments, the vector is an expression vector.

Some embodiments of the present disclosure relate to a pharmaceutical composition comprising an effective antitumor amount of a population of human T or NK cells. The human T or NK cells of the population include human T or NK cells that include the nucleic acid sequence as described above.

Some embodiments of the present disclosure relate to a method of treating the tumor in a human patient, the method comprising administering to the human patient the pharmaceutical composition as described above.

In some embodiments, the tumor is a breast tumor, and the organ lineage antigen comprises PRLR.

In some embodiments, the tumor is a colorectal tumor, and the organ lineage antigen comprises at least one of CLCA1, MUC12, GUCY2C, or GPR35.

In some embodiments, the tumor is a gastric tumor, and the organ lineage antigen comprises CR1L and/or MUC17.

In some embodiments, the tumor is an esophageal tumor, and the organ lineage antigen comprises at least one of TMPRSS11B, MUC21, or TMPRSS11E.

In some embodiments, the tumor is a bladder carcinoma, and the organ lineage antigen comprises CD207.

In some embodiments, the tumor is a pancreatic tumor, and the organ lineage antigen comprises SLC30A8 and/or CFC1.

In some embodiments, the tumor is a cervical tumor, and the organ lineage antigen comprises SLC12A3 and/or SSTR1.

In some embodiments, the tumor is an ovary tumor, and the organ lineage antigen comprises GPR27 and/or FZD10.

In some embodiments, the tumor is a thyroid tumor, and the organ lineage antigen comprises TSHR.

Some embodiments relate to a pharmaceutical composition comprising human T cells. The human T cells may include a nucleic acid sequence encoding a CAR (CAR), the CAR comprises an antigen binding domain, a transmembrane domain, a costimulatory signaling region, and a CD3 zeta signaling domain. The antigen binding domain may include one of amino acid sequences of SEQ ID NOs: 2, 5, 8, 11, 14, 17, or 20.

In some embodiments, the antigen binding domain comprises the amino acid sequence of SEQ ID NO: 2 and binds to Frizzled family receptor 10 (FZD10).

In some embodiments, the antigen binding domain comprises the amino acid sequence of SEQ ID NO: 5 and binds to Thyroid stimulating hormone receptor (TSHR).

In some embodiments, the CAR comprises the amino acid sequence of SEQ ID NO: 8 and binds to prolactin receptor (PRLR).

In some embodiments, the antigen binding domain comprises the amino acid sequence of SEQ ID NO: 11 and binds to Mucin 17 (Muc17).

In some embodiments, the antigen binding domain comprises the amino acid sequence of SEQ ID NO: 14 and binds to Guanylate cyclase 2C (GUCY2C).

In some embodiments, the antigen binding domain comprises the amino acid sequence of SEQ ID NO: 17 and binds to Langerin or Cluster of Differentiation 207 (CD207).

In some embodiments, the antigen binding domain comprises the amino acid sequence of SEQ ID NO: 20 and binds to PRLR.

In some embodiments, the CAR comprises the amino acid sequence of SEQ ID NO: 42 and binds to PRLR.

In some embodiments, the CAR comprises at least one of the amino acid sequences of SEQ ID NO: 36-42.

Some embodiments relate to a method for stimulating a T cell-mediated immune response to a cell population expressing an antigen. The method may include contacting the cell population with an effective amount of human T cells comprising a nucleic acid sequence encoding a CAR (CAR). The CAR may include an antigen binding domain, a transmembrane domain, a costimulatory signaling region, and a CD3 zeta signaling domain. The antigen binding domain may include one of amino acid sequences of SEQ ID NOs: 2, 5, 8, 11, 14, 17, or 20.

In some embodiments, the antigen binding domain comprises the amino acid sequence of SEQ ID NO: 2, and the antigen is FZD10.

In some embodiments, the antigen binding domain comprises the amino acid sequence of SEQ ID NO: 5, and the antigen is TSHR.

In some embodiments, the CAR comprises the amino acid sequence of SEQ ID NO: 8, and the antigen is PRLR.

In some embodiments, the antigen binding domain comprises the amino acid sequence of SEQ ID NO: 11, and the antigen is Muc17.

In some embodiments, the antigen binding domain comprises the amino acid sequence of SEQ ID NO: 14, and the antigen is GUCY2C.

In some embodiments, the antigen binding domain comprises the amino acid sequence of SEQ ID NO: 17, and the antigen is CD207.

In some embodiments, the antigen binding domain comprises the amino acid sequence of SEQ ID NO: 20, and the antigen is PRLR.

In some embodiments, the CAR comprises the amino acid sequence of SEQ ID NO: 42, and the antigen is PRLR.

In some embodiments, the CAR comprises at least one of the amino acid sequences of SEQ ID NO: 36-42.

Some embodiments relate to a method for providing an anti-tumor immune response in a subject. For example, the method relates to stimulating (i.e., eliciting) an anti-tumor immune response in a subject. The method may comprise administrating to the subject an effective amount of a pharmaceutical composition comprising a population of human T cell comprising a nucleic acid sequence encoding a CAR (CAR). The CAR may comprise an antigen binding domain, a transmembrane domain, a costimulatory signaling region, and a CD3 zeta signaling domain. The antigen binding domain binds to a mark of non-essential tissues (e.g., a prolactin receptor).

In some embodiments, the anti-tumor immune response elicited by the CAR T cells may be an active or a passive immune response. In certain embodiments, the CAR T cells mediated immune response may be part of an adoptive immunotherapy approach in which CAR T cells induce an immune response specific to the antigen binding domain in the CAR. For example, T cells that express an anti-PRLR CAR elicits an immune response specific against cells expressing prolactin receptors. In some embodiments, the anti-tumor immune response comprises a reduction in tumor burden on the subject with the tumor (e.g., breast cancer).

In some embodiments, the antigen binding domain is an antibody, a ligand, or an antigen-binding fragment thereof.

In some embodiments, the antigen-binding fragment is a Fab or a scFv.

In some embodiments, antigen binding domain comprises amino acid sequences of SEQ ID NOs: 8.

In some embodiments, the CAR comprises an amino acid sequence of SEQ ID NO: 42.

In some embodiments, the costimulatory signaling region comprises the intracellular domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

Table 1 below lists multiple tumor and organ lineage antigens as well as corresponding genes.

| | | Gene | Corresponding |
|---|---|---|---|
| Cancer types | Gene | Abbreviation | Protein/antigen |
| Breast Cancer | Prolactin receptor | PRLR | PRLR |
| colorectal Cancer | Chloride channel accessory 1 | CLCA1 | CLCA1 |
| colorectal Cancer | Mucin 12 | MUC12 | MUC12 |
| colorectal Cancer | Guanylate cyclase 2C | GUCY2C | GUCY2C |
| colorectal Cancer | G protein-coupled receptor 35 | GPR35 | GPR35 |
| Gastric Cancer | Complement component | CRIL | CR1L |
| | (3b/4b) receptor 1-like | | |
| Gastric Cancer | Mucin 17, cell surface associated | MUC17 | MUC17 |
| esophageal Cancer | Transmembrane protease, serine 11B | TMPRSS11B | TMPRSS11B |
| esophageal Cancer | Mucin 21 | MUC21 | MUC21 |
| esophageal Cancer | Transmembrane protease, serine 11E | TMPRSS11E | TMPRSS11E |
| bladder Cancer | CD207 | CD207 | CD207 |
| pancreatic Cancer | Solute carrier family 30 | SLC30A8 | SLC30A8 |
| | (zinc transporter), | | |
| | member 8 | | |
| pancreatic Cancer | Cripto, FRL- 1, cryptic family 1 | CFC1 | CFC1 |
| Cervical Cancer | Solute carrier family 12 | SLC12A3 | SLC12A3 |
| | (sodium/chloride transporters) | | |
| | member 3 | | |
| Cervical tumor | Somatostatin receptor 1 | SSTR1 | SSTR1 |
| Ovary tumor | G protein-coupled receptor 27 | GPR27 | GPR27 |
| Ovary tumor | Frizzled family receptor 10 | FZD10 | FZD10 |
| Thyroid Tumor | Thyroid stimulating hormone receptor | TSHR | TSHR |

### EXAMPLES

The present disclosure is further described with reference to the following examples. These examples are provided for purposes of illustration only and are not intended to be limiting unless otherwise specified. Thus, the present disclosure should in no way be construed as being limited to the following examples, but rather, should be construed to encompass any and all variations which become evident as a result of the teaching provided herein.

### Construction of Antigen-Expressed K562 Cell Lines

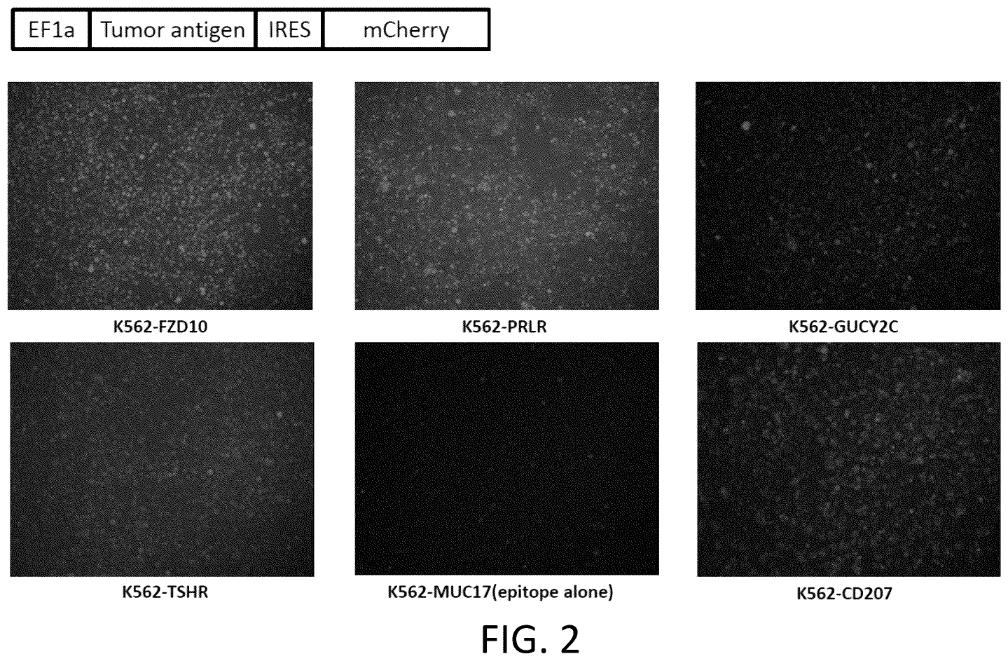

K562 cells were transduced with lentivirus including nucleic acid sequences encoding various antigens (FIG. 1) to establish target tumor cell lines. The lentivirus included the IRES-mCherry (red) construct, which encodes red fluorescence for confirmation of antigen expression. Red fluorescent signals were observed from these cell lines, indicating that target solid tumor cell lines were successfully established (FIG. 2). Techniques of construction of cell lines may be found at “Chimeric Receptors Containing CD137 Signal Transduction Domains Mediate Enhanced Survival of T Cells and Increased Antileukemic Efficacy In Vivo Molecular Therapy vol. 17 no. 8, 1453-1464 August 2009,” which is incorporated herein by reference. K562 cells were obtained from American Type Culture Collection (ATCC).

### Construction of CAR T Cells

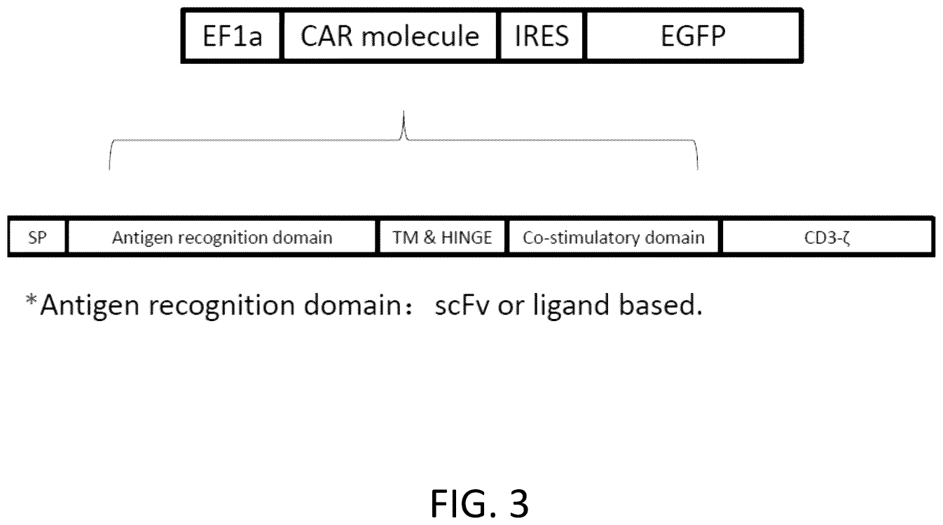

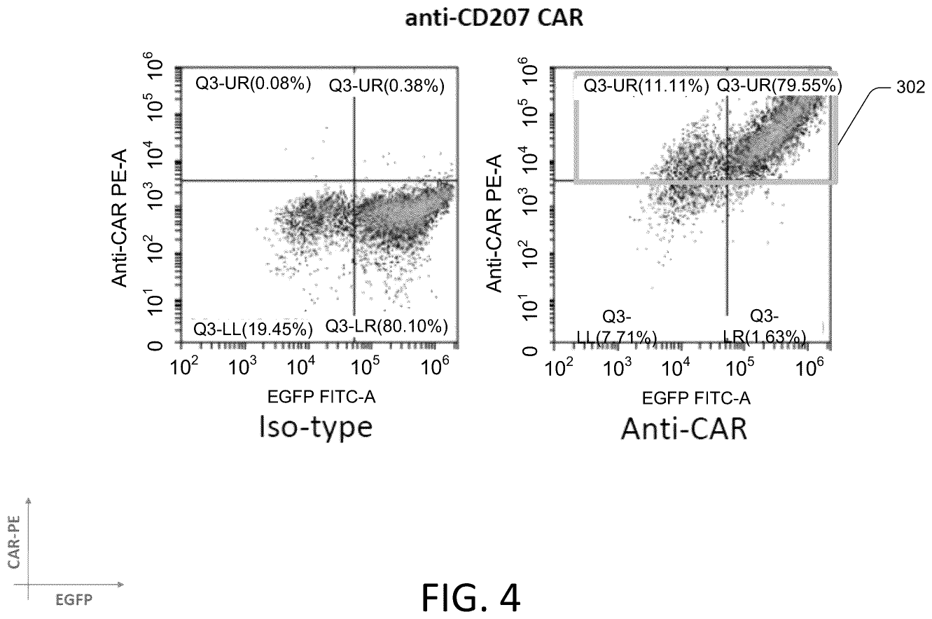

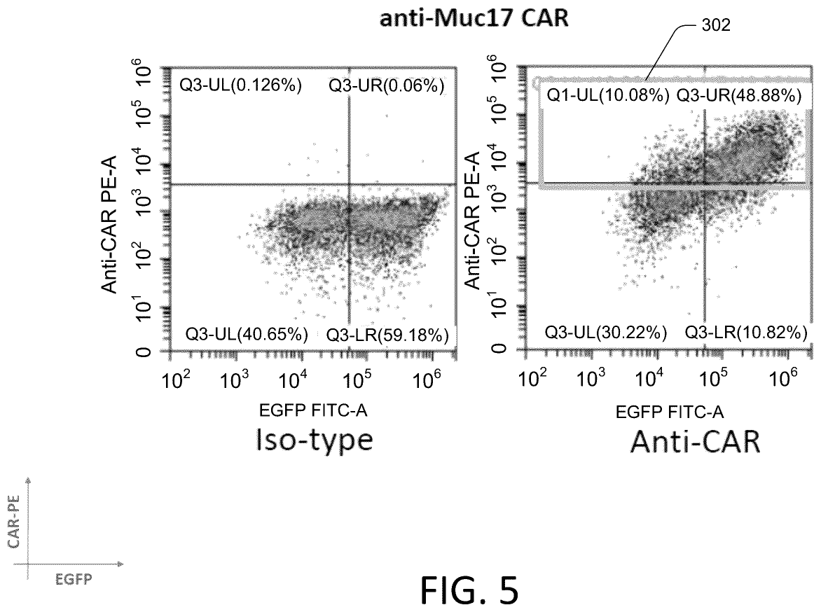

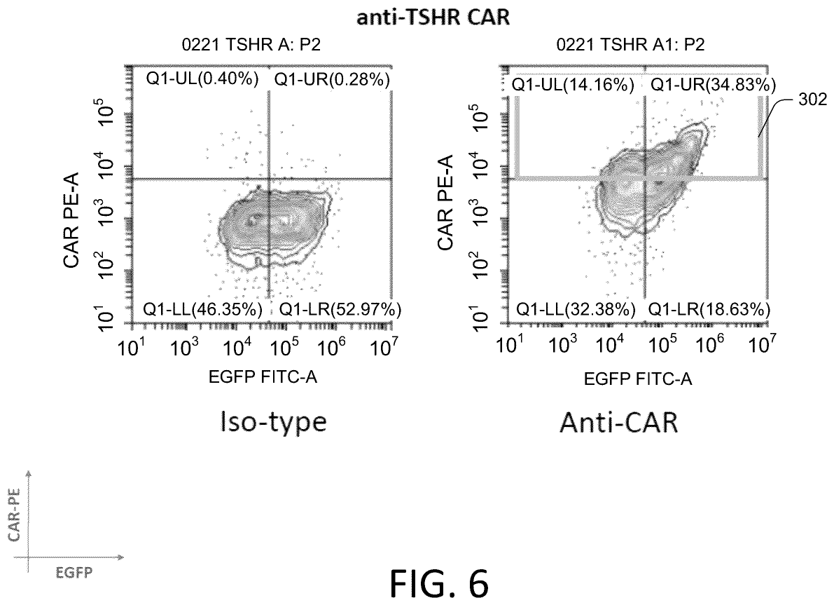

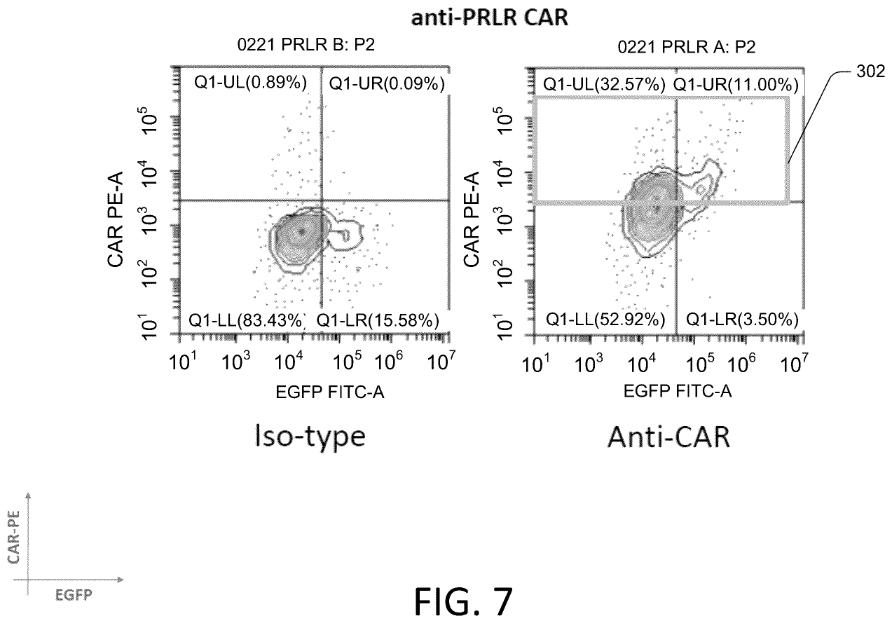

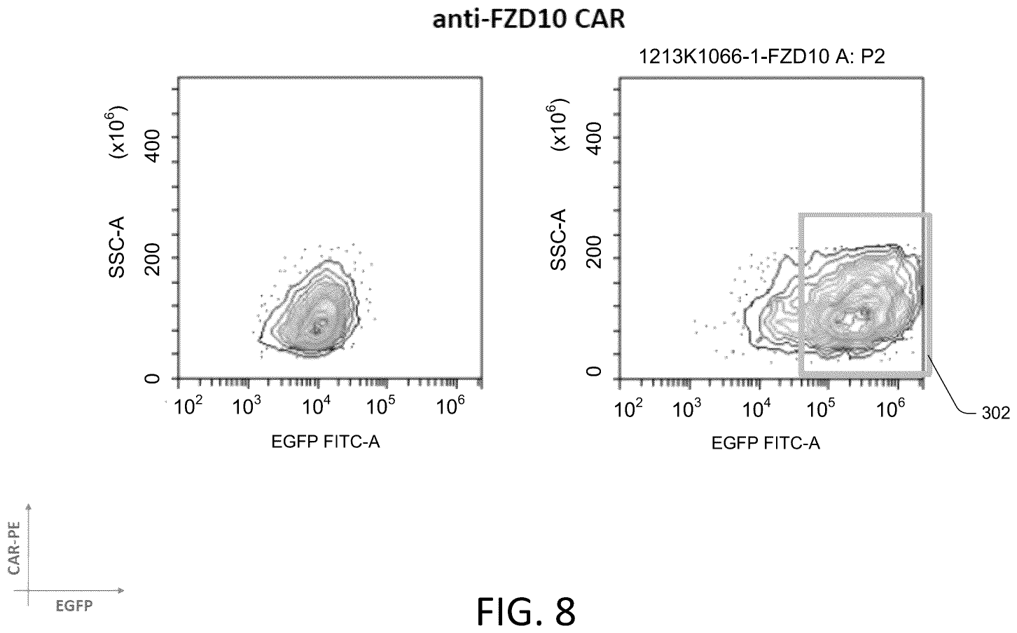

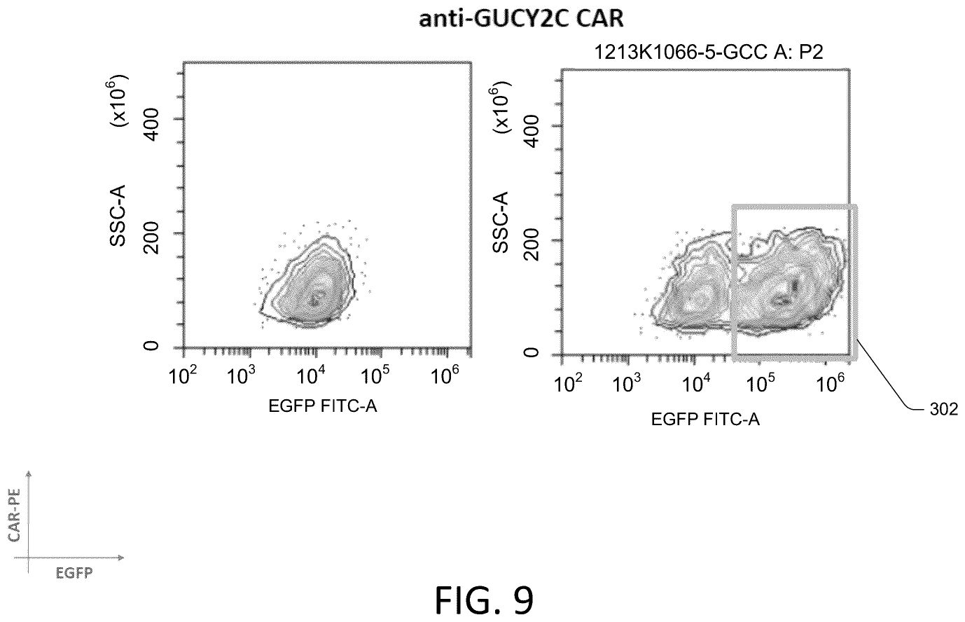

Primary T cells were transduced with lentivirus including various CARs to establish different CAR T cell lines targeting different antigens listed in FIG. 1. These cells were obtained from healthy human donors. As illustrated in FIG. 3, the lentivirus included nucleic acid sequence encoding CAR molecules, respectively, and further included the IRES-mCherry (green) construct, which encodes green fluorescence for confirmation of CAR expression. Taking anti-CD207/anti-Muc17 CAR T cell as examples in FIG. 3A, and expression of CARs was measured to confirm that CAR T-cell lines express specific anti-antigen molecules (See above boxes 302) in FIGS. 4-9. Techniques related to cell cultures, construction of lentiviral vectors, and flow cytometry may be found in “Treatment of Advanced Leukemia in Mice with mRNA-Engineered T Cells, HUMAN GENE THERAPY 22:1575-1586 (December 2011)”, which is incorporated herein by reference.

Table 2 below lists various sequence identifiers and their sequences for establishing various anti-antigen CAR T cells.

| SEQ ID NO: | Identifier | Target tumors |
|---|---|---|
| 2 | scFv FZD10 | Ovary tumor |
| 5 | scFv TSHR | Thyroid Tumor |
| 8 | scFv PRLR | Breast cancer |
| 11 | scFv Muc17 | Gastric Cancer |
| 14 | scFv GUCY2C | colorectal Cancer |
| 17 | scFv CD207 | bladder Cancer |
| 36 | CAR FZD10 | Ovary tumor |
| 37 | CAR TSHR | Thyroid Tumor |
| 38 | CAR PRLR | Breast cancer |
| 39 | CAR Muc17 | Gastric Cancer |
| 40 | CAR GUCY2C | colorectal Cancer |
| 41 | CAR CD207 | bladder Cancer |
| 42 | CAR Prolactin | Breast cancer |
| 44 | CAR modified Prolactin | Breast cancer |

### IFN-γ Release in Co-Cultivation Assays

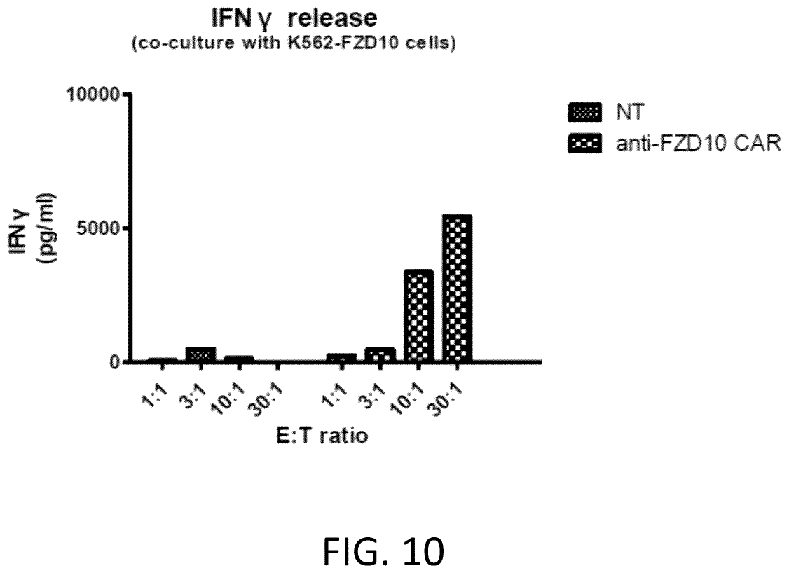

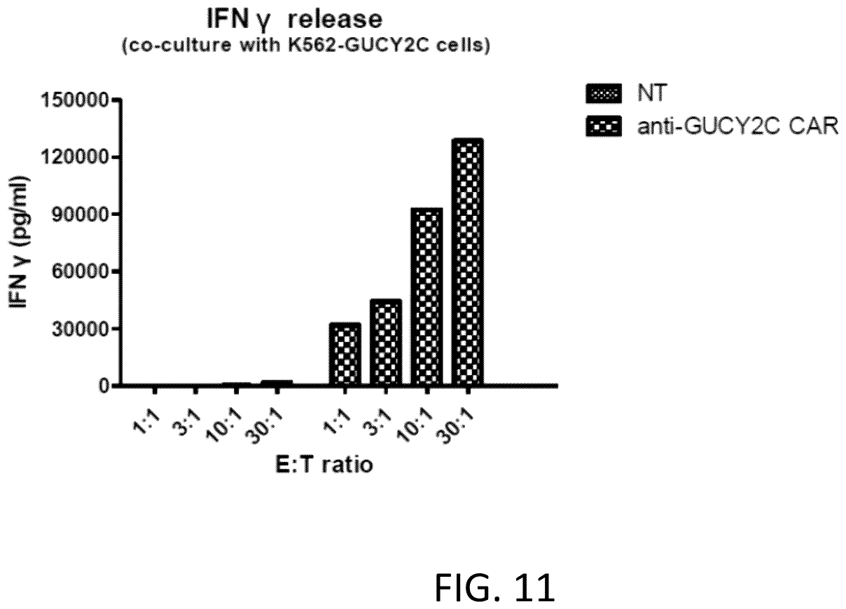

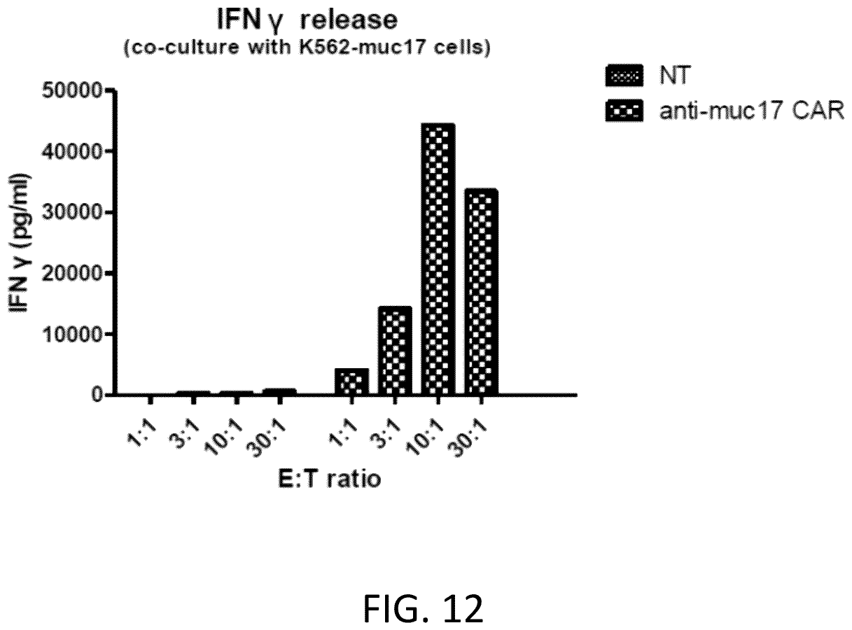

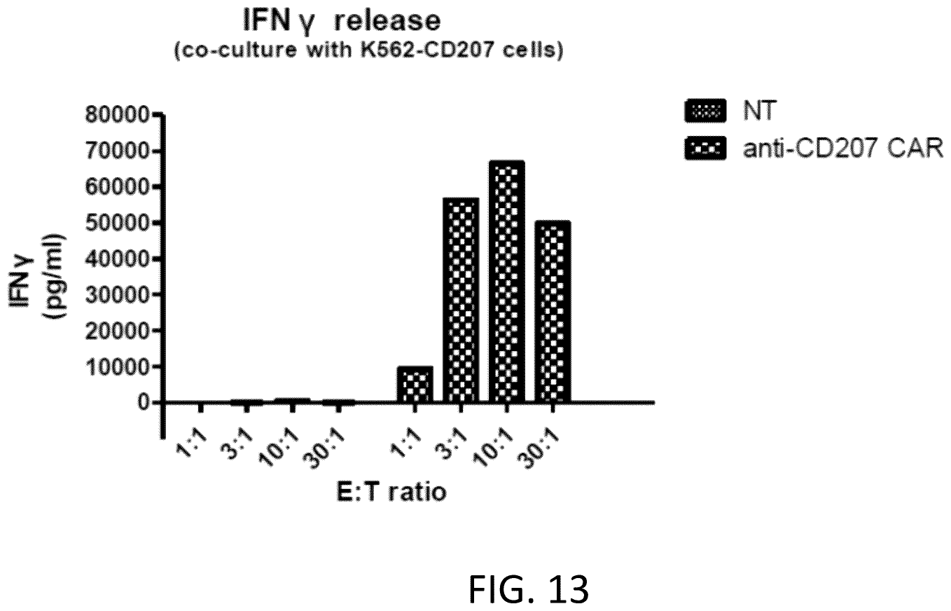

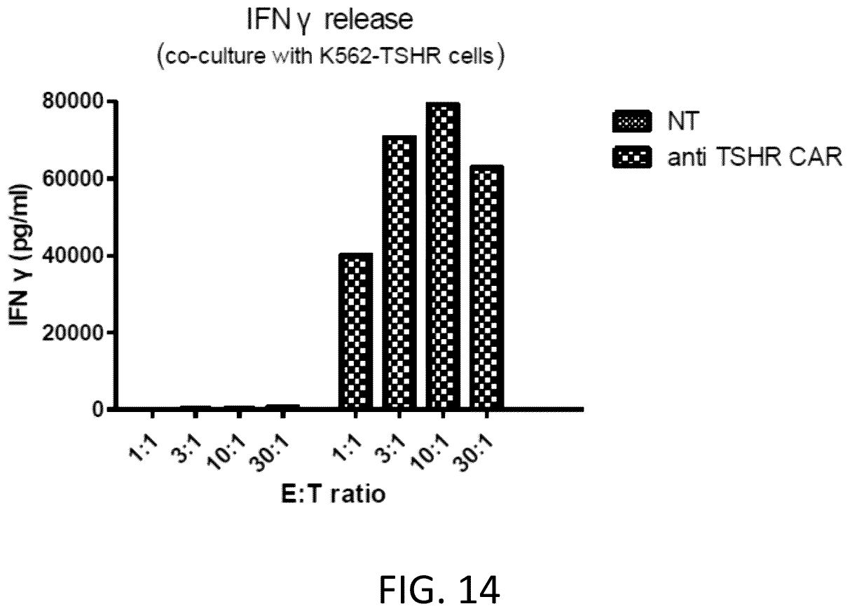

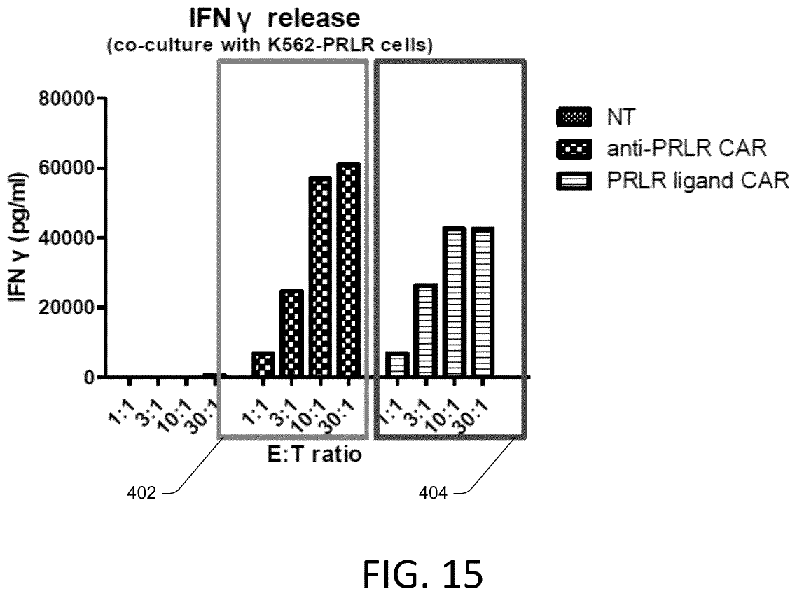

Each type of CAR T cells and the corresponding type of antigen-expressed K562 cells were cocultured (See, Table 2), and CAT T cells' responses induced by the antigen-express K562 cells were measured. A ratio of E:T 1:1/3:1/10:1/30:1 (i.e., CAR T cells: target tumor cells) of CAR T cells and target tumor cells were cocultured for 24 hours. The supernatant was collected then, and release of IFN-γ was measured. Various levels of IFN-γ release were observed when CAR T cells and their corresponding antigen-express K562 cells were co-cultured. IFN-γ release is not obvious when the CAR T cells and wild-type K562 were co-cultured. This observation shows that the CAR T cells specifically identify the corresponding antigen-express K562 cells and attack these cells by releasing IFN-γ (see FIGS. 10-15). As illustrated in box 404 of FIG. 4B, T cells including prolactin-CAR also recognize target tumor cells expressing PRLR and release IFN-γ in response to co-culturing the prolactin-CAR T cells and the antigen-express K562 cells. As compared to box 402 in FIG. 15 (scFv anti-PRLR CAR T cells), prolactin-CAR T cells achieve a similar effect. Techniques related to cell cultures, construction of cytotoxic T-lymphocyte assay may be found in “Control of large, established tumor xenografts with genetically retargeted human T cells containing CD28 and CD137 domains. 3360-3365 PNAS Mar. 3, 2009, vol. 106 no. 9”, which is incorporated herein by reference.

### CAT T Cell Killing Assay

CAR T cell killing assays were conducted to measure the effectiveness of CAR T cells. Primary T cells were obtained from blood samples of healthy human donors. These T cells were transduced with a nucleic acid sequence encoding various CARs (See Table 2 and FIG. 1), respectively, and CAR expression on T-cells was measured using flow cytometry techniques.

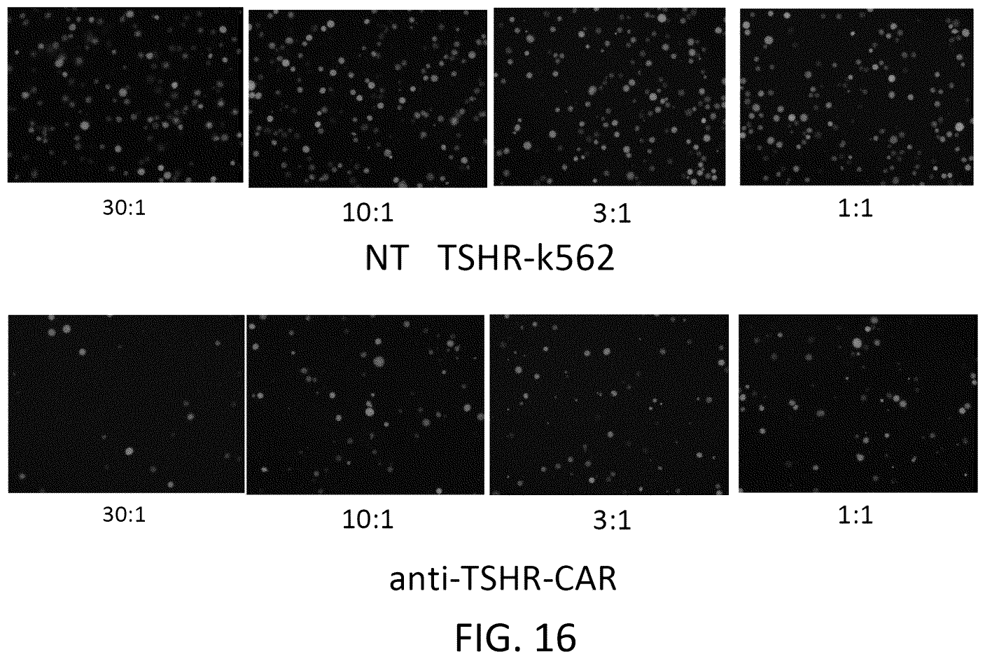

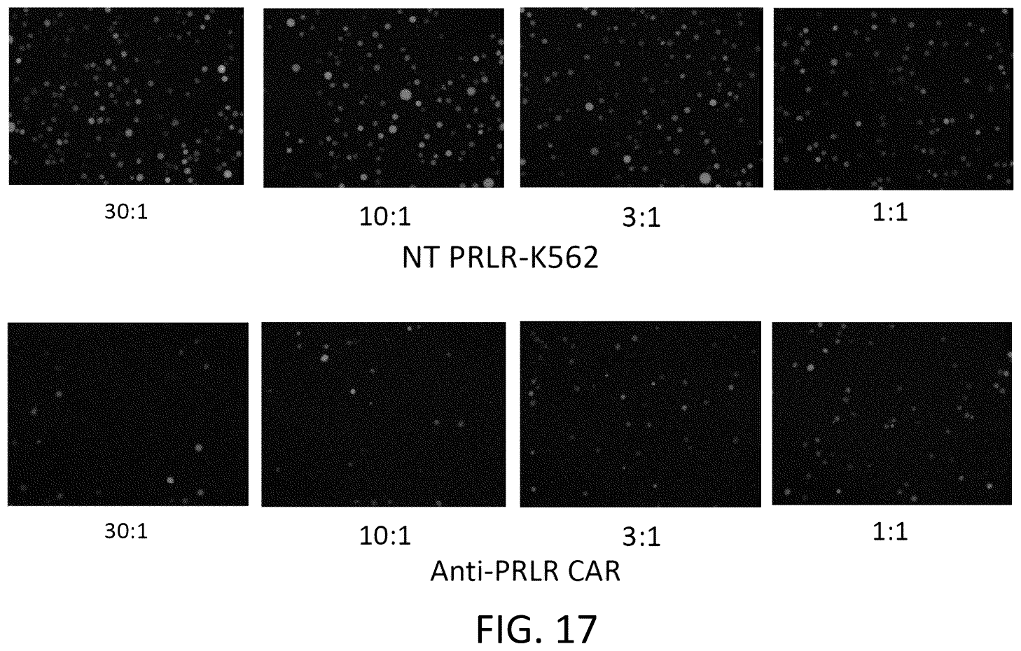

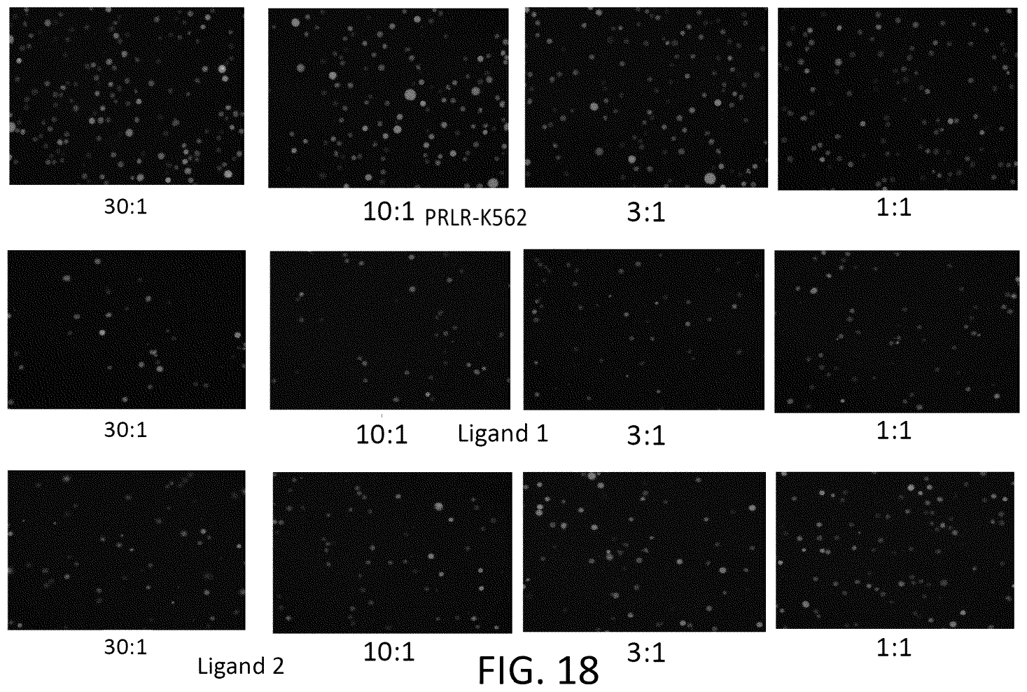

K562 cells were transduced with nucleic acid sequences encoding corresponding human antigens (See FIG. 1), respectively, and antigen expression was measured using flow cytometry techniques. Further antigen-expression K562 cells were transduced with a nucleic acid sequence encoding fluorescent proteins (RFP) for killing assay analysis. Various CAR T cells were incubated with corresponding K562 cells for 24 hours in various E:T ratios (30:1, 10:1, 3:1, 1:1), and red fluorescence signals from cocultured cells were observed. For example, CAR T cells expressing anti-THSR CAR (SEQ ID NO: 5) were co-cultured with K562 expressing human THSR (SEQ ID NO: 26) for at least five days. As compared with normal T cells, CAR T cells significantly reduced numbers of antigen-expression K562 cells. Examples of anti-PRLR CAR, anti-THSR CAR, and Prolactin CAR T cells were provided in FIGS. 16-18. In these examples, red fluorescence signals of cocultured cells were observed at day five after co-culturing the CAR T cells and the corresponding antigen-expression K562 cells.

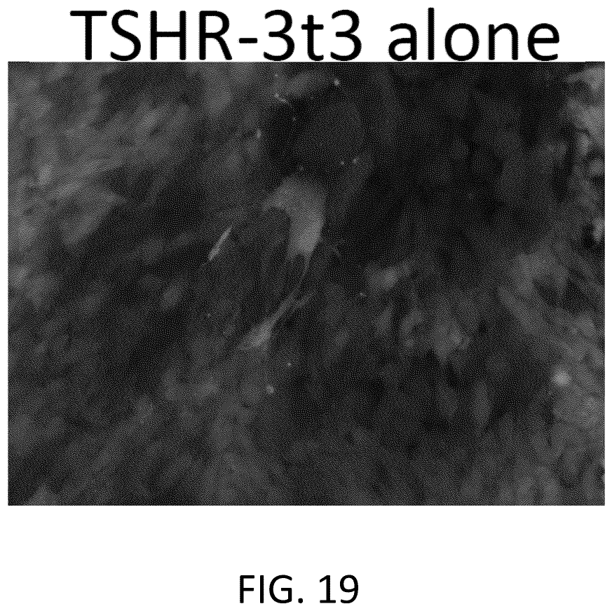

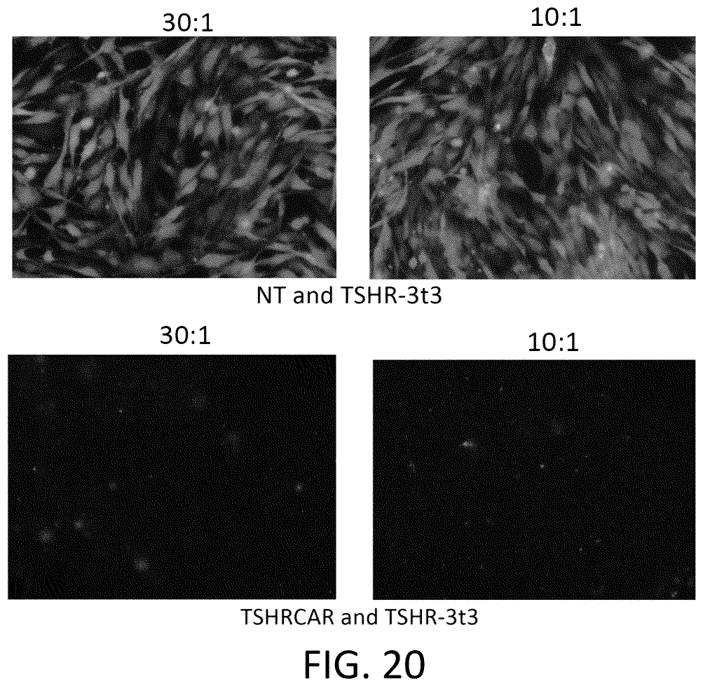

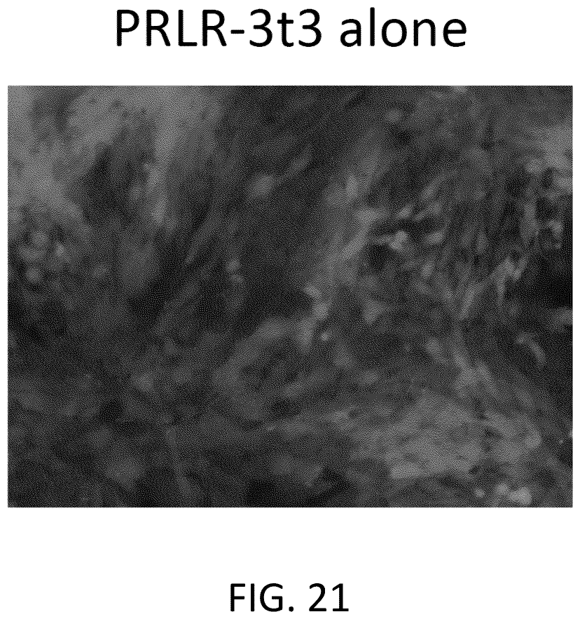

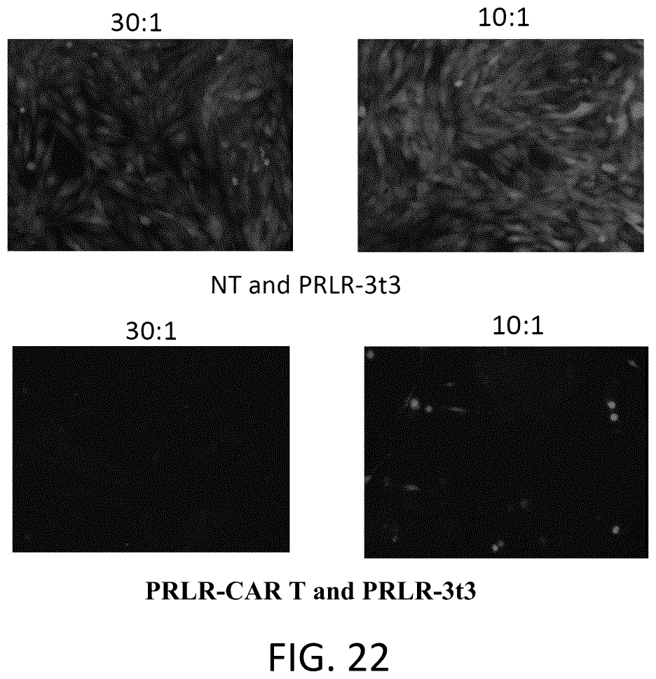

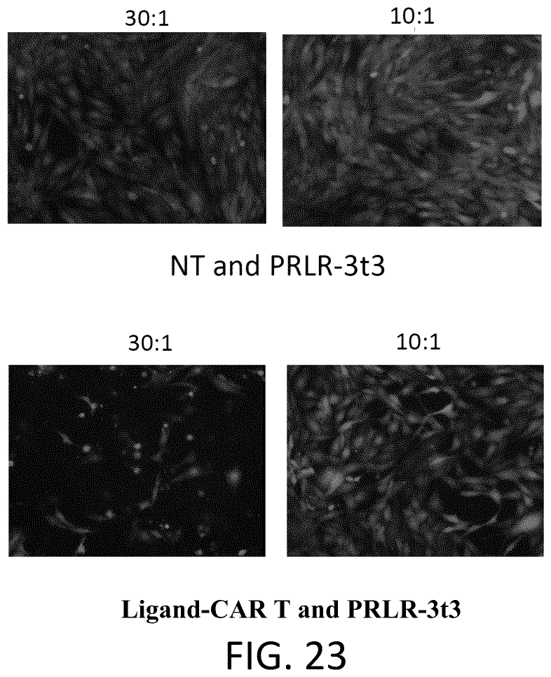

The car T cell killing analysis was further performed using 3T3 murine fibroblasts (ATCC). 3T3 cells were transduced with various human antigens (See FIG. 1), and antigen expression on 3T3 cells was measured using flow cytometry techniques. Further, 3T3 cells were transduced with nucleic acid sequence encoding fluorescent proteins (RFP) for killing assay analysis. 3T3 cells expressing human target antigens/RFP and corresponding CAR T cells were cocultured at an E:T 30:1 or 10:1, respectively, and then fluorescent signals were observed from the cocultured cells for at least five days. As compared with normal T cells, CAR T cells significantly reduced numbers of antigen-expression 3T3 cells. Examples of anti-PRLR CAR, anti-PRLR CAR, and Prolactin CAR T cells were provided in FIGS. 19-23. In these examples, red fluorescence signals of cocultured cells were observed at day five after co-culturing the CAR T cells and the corresponding antigen-expression 3T3 cells.

### In Vivo Anti-Tumor Activity

Heterotransplantation of human cancer cells or tumor biopsies into immunodeficient rodents (xenograft models) has, for the past two decades, constituted the major preclinical screen for the development of novel cancer therapeutics (Song et al., Cancer Res. PMC 2014 Aug. 21, and Morton et al., Nature Protocols, 2, -247-250 (2007)). To evaluate the anti-tumor activity of CAT T cells in vivo, immunodeficient mice bearing tumor xenografts were to evaluate CAR T's anti-tumor activity.

K562-PRLR-RFP cells were used to establish the immunodeficient mice bearing PRLR tumor xenografts. On day one, K562-PRLR-RFP cells were injected into tail veins of the immunodeficient mice. On day two or three, irradiation was performed on the immunodeficient mice in 2 Gy fractions. On day three, the formation of tumor cells in the immunodeficient mice was observed.

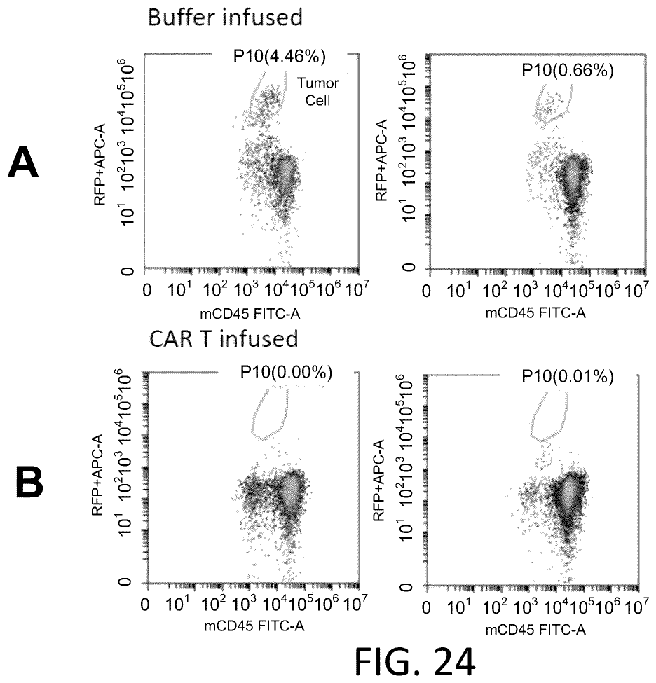

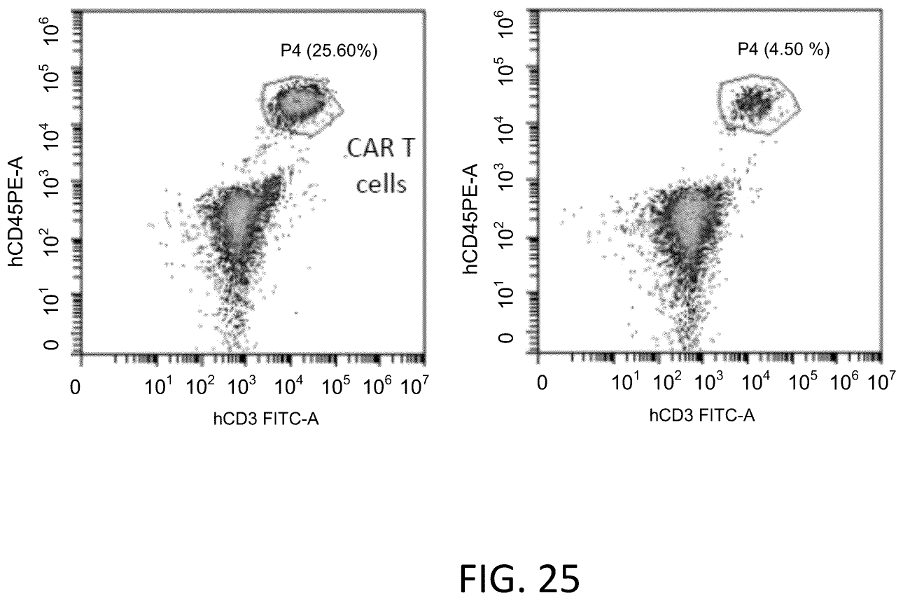

On day three, anti-PRLR human CAR T cells (i.e., anti-PRLR CAR T) were transfused to the immunodeficient mice, and anti-tumor activities were observed in the immunodeficient mice. The anti-PRLR CAR T cells were made by the protocol described in this present disclosure. The presence of K562-PRLR-RFP cells was evaluated using the peripheral blood of the immunodeficient mice by flow cytometry after three or four weeks. In control, the buffer was transfused to the immunodeficient mice, and the immunodeficient mice died within four to six weeks. As for the immunodeficient mice transfused with anti-PRLR CAR T, the K562-PRLR-RFP cells were not observed, and the immunodeficient mice behaved normally. Human CD3 cells were further observed in the immunodeficient mice (FIGS. 24 and 25). It is concluded that CAR T cells have anti-tumor activity in mice. Additional information of the protocol was provided in Table 3 below.

| Tumor cell | K562-PRLR RFP cells |
|---|---|
| Tumor cells transplanted | 5*10\hat{ }5 cells/mouse |
| irradiation | 2Gy |
| CAR T cells infused | 1*10\hat{ }7 cells/mouse |

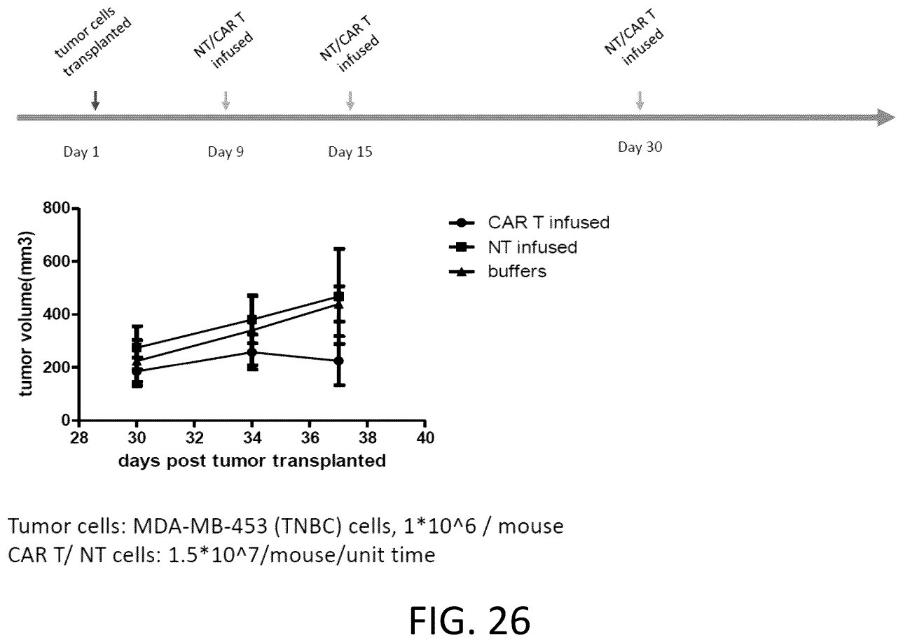

Anti-tumor activity of CAR T cells was further observed in TNBC xenografts in immunodeficient mice. MDA-MB-453 (TNBC, triple-negative breast cancer) cell lines were used to established TNBC xenografts in immunodeficient mice. As illustrated in the upper diagram of FIG. 26 shows, TNBC xenografts in immunodeficient mice were established by subcutaneously transplanting cells of MDA-MB-453 (TNBC) cell lines. After transplantation, 1.5×10⁷ NT/anti-PRLR human CAR T cells (anti-PRLR CAR T) were transfused to each of the mice on Day 9, Day 15, and Day 30 (e.g., 7×10⁵ CAR T cells per gram). Tumor volumes were observed and measured. Tumor volumes were calculated using the equation: V=π/6*a*b*b. “a” is the long axis of the tumor, and “b” is the minor axis. As shown in the lower plot of FIG. 26, in vivo antitumor activity was observed in mice that are transfused with anti-PRLR CAR T.

As described above, the treatment methods described herein can easily be adapted for other species or subjects, such as humans.

Table 4 below lists various sequence identifiers and their sequences.

| SEQ ID NO: | Identifier |
|---|---|
| 1 | SP |
| 2 | scFv FZD10 |
| 3 | 2L-FZD10 |
| 4 | 2H-FZD10 |
| 5 | scFv TSHR |
| 6 | 5L-TSHR |
| 7 | 5H-TSHR |
| 8 | scFv PRLR |
| 9 | 8L-PRLR |
| 10 | 8H-PRLR |
| 11 | scFv Muc17 |
| 12 | 11L-Muc17 |
| 13 | 11H-Muc17 |
| 14 | scFV GUCY2C |
| 15 | 14L-GUCY2C |
| 16 | 14H-GUCY2C |
| 17 | scFv CD207 |
| 18 | 17L-CD207 |
| 19 | 17H-CD207 |
| 20 | Prolactin (ligand) |
| 21 | Hinge & transmembrane domain |
| 22 | Co-stimulatory region |
| 24 | A-FZD10 |
| 25 | A-FZD10 (amino acid) |
| 26 | B-TSHR |
| 27 | B-TSHR (amino acid) |
| 28 | C-PRLR |
| 29 | C-PRLR (amino acid) |
| 30 | D-Muc17 |
| 31 | D-Muc17 (amino acid) |
| 32 | E-GCC |
| 33 | E-GCC (amino acid) |
| 34 | F-CD207 |
| 35 | F-CD207 (amino acid) |
| 36 | CAR FZD10 |
| 37 | CAR TSHR |
| 38 | CAR PRLR |
| 39 | CAR Muc17 |
| 40 | CAR GUCY2C |
| 41 | CAR CD207 |
| 42 | CAR Prolactin |
| 43 | CAR modified Prolactin |
| 44 | Prolactin (mutation) |
| 45 | Hinge & transmembrane domain |

What is claimed is:

1. A method of measuring a cytokine in a T cell-mediated immune response, the method comprising:
contacting a population of cells comprising guanylate cyclase 2C (GUCY2C) comprising the amino acid sequence of SEQ ID NO: 33, with a population of modified T cells comprising a chimeric antigen receptor (CAR) in a medium, the CAR comprising an extracellular domain, a transmembrane domain, and an intracellular domain, the extracellular domain comprising the amino acid sequence SEQ ID NO: 14;
collecting a supernatant from the medium; and
measuring a concentration of the cytokine in the supernatant, the cytokine including INF-γ.

2. The method of claim 1, wherein the population of modified T cells and the population of cells comprising GUCY2C are cocultured at a ratio of effector cells to target cells is 1:1, 1:3, 1:10, or 1:30.

3. The method of claim 1, wherein the population of cells comprising GUCY2C is a population of K562 cells transfected to express GUCY2C.

4. The method of claim 1, wherein the extracellular domain of the CAR comprises a scFv antigen binding domain.

5. The method of claim 1, wherein the intracellular domain of the CAR comprises a CD3-zeta (CD37) signaling domain.

6. The method of claim 1, wherein the supernatant is collected after coculturing the populations of cells for 24 hours.

7. The method of claim 1, further comprising measuring a concentration of IFN-γ released when the population of modified T cells is cultured with a control population of cells not expressing GUCY2C, and comparing the amounts of IFN-γ released in each case.

8. The method of claim 7, wherein IFN-γ release is observed when the population of modified T cells is cultured with the population of cells comprising GUCY2C and IFN-γ release is not observed when the population of modified T cells is cultured with the control population of cells.

9. The method of claim 1, wherein the concentration of the cytokine is measured using an ELISA assay.

10. The method of claim 1, wherein the intracellular domain further comprises a costimulatory domain.

11. The method of claim 10, wherein the costimulatory domain is a 4-1BB costimulatory domain.

12. The method of claim 1, wherein the CAR transduces a signal in response to binding of the extracellular domain to GUCY2C on the surface of the population of cells.

13. The method of claim 1, wherein transduced T cells expressing the chimeric antigen receptor release greater than 100 pg/ml of IFN-γ.

14. The method of claim 1, wherein the medium comprises interleukin-2 (IL-2) or interleukin-15 (IL-15).

15. The method of claim 1, wherein the population of cells is contacted with the population of transduced T cells for at least 18 hours.

16. The method of claim 1, wherein the concentration of IFN-γ measured is at least 2-fold greater than a baseline level of IFN-γ release by non-transduced T cells.

17. The method of claim 1, wherein IFN-γ release is lower when the CAR T cells are cultured with non-transduced K562 cells compared to K562 cells expressing GUCY2C.

18. The method of claim 1, wherein the CAR T cells exhibit IFN-γ release in response to tumor cells engineered to express GUCY2C but not in response to normal tissues intrinsically expressing GUCY2C.

19. A method of treating patient with cancer, wherein the method comprises contacting a population of cells comprising guanylate cyclase 2C (GUCY2C) comprising the amino acid sequence of SEQ ID NO: 33 in a patient with a population of modified T cells comprising a chimeric antigen receptor (CAR), the CAR comprising an extracellular domain, a transmembrane domain, and an intracellular domain, the extracellular domain comprising the amino acid sequence SEQ ID NO: 14; and
detecting release of one or more cytokines in the patient;
wherein release of one or more cytokines indicates activation of T cell-immune response for treating the patient.

20. The method of claim 19, wherein the one or more cytokines include INF-γ.

21. The method of claim 19, wherein the cancer is colorectal cancer.


SEQUENCE LISTING (45 sequences)

SEQ ID NO: 1 - protein, 21 aa, synthetic construct
  REGION 1..21: note = synthetic
MALPVTALLLPLALLLHAARP

SEQ ID NO: 2 - protein, 237 aa, synthetic construct
  REGION 1..237: note = synthetic
DIQMTQSPASLSVSVGETVTITCRASENIYSNLAWYQQKQGKSPQLLVYVATNLADGVPSRFSGSGSGTQYSLKINSLQSEDFGSYYCQHFWGTPYTFGGGTKLGGGGSGGGGSGGGGSEVQLQQSGAELVKPGASVKLSCTASGFNINDTYMHWVKQRPEQGLEWIGRIDPANGNTKYDPKFQGKATITADTSSNTAYLQLSSLTSEDTAVYYCARGARGSRFAYWGQGTLVTVSA

SEQ ID NO: 3 - protein, 104 aa, synthetic construct
  REGION 1..104: note = synthetic
DIQMTQSPASLSVSVGETVTITCRASENIYSNLAWYQQKQGKSPQLLVYVATNLADGVPSRFSGSGSGTQYSLKINSLQSEDFGSYYCQHFWGTPYTFGGGTKL

SEQ ID NO: 4 - protein, 118 aa, synthetic construct
  REGION 1..118: note = synthetic
EVQLQQSGAELVKPGASVKLSCTASGFNINDTYMHWVKQRPEQGLEWIGRIDPANGNTKYDPKFQGKATITADTSSNTAYLQLSSLTSEDTAVYYCARGARGSRFAYWGQGTLVTVSA

SEQ ID NO: 5 - protein, 244 aa, synthetic construct
  REGION 1..244: note = synthetic
QSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVLGGGGSGGGGSGGGGSEVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSS

SEQ ID NO: 6 - protein, 110 aa, synthetic construct
  REGION 1..110: note = synthetic
QSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVL

SEQ ID NO: 7 - protein, 119 aa, synthetic construct [sequence omitted]

SEQ ID NO: 8 - protein, 252 aa, synthetic construct
  REGION 1..252: note = synthetic
DIVLTQSPASLAVSLGQGATISCRASKSVSTSGYTYMHWYQQKPGQPPKLLIYLASNLESGVPARFSGSGSGTDFTLNIHPVEEEDAATYYCQHSGELPPSFGGGTKLEIKRAGGGGSGGGGSGGGGSEVQLVESGGDLVKPGGSLKLSCAVSGFTFSSYGMSWVRQTPDKRLEWVATVSSGGTYTYYPDSVKGRFTISRDNAKNTLYLQMSSLKSEDSAMYYCARHRGNYYATYYYAMDYWGQGTSVTVSS

SEQ ID NO: 9 - protein, 113 aa, synthetic construct
  REGION 1..113: note = synthetic
DIVLTQSPASLAVSLGQGATISCRASKSVSTSGYTYMHWYQQKPGQPPKLLIYLASNLESGVPARFSGSGSGTDFTLNIHPVEEEDAATYYCQHSGELPPSFGGGTKLEIKRA

SEQ ID NO: 10 - protein, 124 aa, synthetic construct
  REGION 1..124: note = synthetic
EVQLVESGGDLVKPGGSLKLSCAVSGFTFSSYGMSWVRQTPDKRLEWVATVSSGGTYTYYPDSVKGRFTISRDNAKNTLYLQMSSLKSEDSAMYYCARHRGNYYATYYYAMDYWGQGTSVTVSS

SEQ ID NO: 11 - protein, 243 aa, synthetic construct
  REGION 1..243: note = synthetic
DVQITQSPSYLAASPGETITINCRASKSISKYLAWYQEKPGKTNKLLIYSGSTLQSGIPSRFSGSGSGTDFTLTISSLEPEDFAMYYCQQHHEYPYTFGGGTKLEIKGGGGSGGGGSGGGGSQVQLQQPGAELVRPGASVKLSCKASGYTFTTYWMNWVKQRPEQGLEWIGRIDPYDSETHYNQKFKDKAILTVDKSSSTAYMQLSSLTSEDSAVYYCARGPYYGTNPWFPYWGQGTLVTVSS

SEQ ID NO: 12 - protein, 107 aa, synthetic construct
  REGION 1..107: note = synthetic
DVQITQSPSYLAASPGETITINCRASKSISKYLAWYQEKPGKTNKLLIYSGSTLQSGIPSRFSGSGSGTDFTLTISSLEPEDFAMYYCQQHHEYPYTFGGGTKLEIK

SEQ ID NO: 13 - protein, 121 aa, synthetic construct
  REGION 1..121: note = synthetic
QVQLQQPGAELVRPGASVKLSCKASGYTFTTYWMNWVKQRPEQGLEWIGRIDPYDSETHYNQKFKDKAILTVDKSSSTAYMQLSSLTSEDSAVYYCARGPYYGTNPWFPYWGQGTLVTVSS

SEQ ID NO: 14 - protein, 241 aa, synthetic construct
  REGION 1..241: note = synthetic
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 15 - protein, 107 aa, synthetic construct
  REGION 1..107: note = synthetic
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIK

SEQ ID NO: 16 - protein, 119 aa, synthetic construct
  REGION 1..119: note = synthetic
QVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 17 - protein, 246 aa, synthetic construct
  REGION 1..246: note = synthetic
DVVMTQTPLSLPVRLGDQASISCRSSQSLVHSNGNTYLHWYLQKPGQSPKLLIYKVSNRFSGVPDRFSGSGSGTNFTLKISRVEAEDLGLYFCSQSTHVPYTFGGGTKLEIKRAGGGGSGGGGSGGGGSQVQLRQSGPELVKPGASVKMSCKASGYTFTDYVISWVKQRTGQGLEWIGDIYPGSGYSFYNENFKGKATLTADKSSTTAYMQLSSLTSEDSAVYFCATYYNYPFAYWGQGTLVTVSA

SEQ ID NO: 18 - protein, 114 aa, synthetic construct
  REGION 1..114: note = synthetic
DVVMTQTPLSLPVRLGDQASISCRSSQSLVHSNGNTYLHWYLQKPGQSPKLLIYKVSNRFSGVPDRFSGSGSGTNFTLKISRVEAEDLGLYFCSQSTHVPYTFGGGTKLEIKRA

SEQ ID NO: 19 - protein, 117 aa, synthetic construct
  REGION 1..117: note = synthetic
QVQLRQSGPELVKPGASVKMSCKASGYTFTDYVISWVKQRTGQGLEWIGDIYPGSGYSFYNENFKGKATLTADKSSTTAYMQLSSLTSEDSAVYFCATYYNYPFAYWGQGTLVTVSA

SEQ ID NO: 20 - protein, 199 aa, synthetic construct
  REGION 1..199: note = synthetic
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 21 - protein, 69 aa, synthetic construct
  REGION 1..69: note = synthetic
AKPTTTPAPRPPTPAPTIASQPLSLRPEACRPAAGGAVHTRGLDFACDIYIWAPLAGTCGVLLLSLVIT

SEQ ID NO: 22 - protein, 42 aa, synthetic construct
  REGION 1..42: note = synthetic
KRGRKKLLYIFKQPFMRPVQTTQEEDGCSCRFPEEEEGGCEL

SEQ ID NO: 23 - protein, 112 aa, synthetic construct
  REGION 1..112: note = synthetic
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - DNA, 1746 bp, synthetic construct
  misc_feature 1..1746: note = synthetic
  source 1..1746: mol_type = other DNA
atgcagcgcccgggcccccgcctgtggctggtcctgcaggtgatgggctcgtgcgccgccatcagctccatggacatggagcgcccgggcgacggcaaatgccagcccatcgagatcccgatgtgcaaggacatcggctacaacatgactcgtatgcccaacctgatgggccacgagaaccagcgcgaggcagccatccagttgcacgagttcgcgccgctggtggagtacggctgccacggccacctccgcttcttcctgtgctcgctgtacgcgccgatgtgcaccgagcaggtctctacccccatccccgcctgccgggtcatgtgcgagcaggcccggctcaagtgctccccgattatggagcagttcaacttcaagtggcccgactccctggactgccggaaactccccaacaagaacgaccccaactacctgtgcatggaggcgcccaacaacggctcggacgagcccacccggggctcgggcctgttcccgccgctgttccggccgcagcggccccacagcgcgcaggagcacccgctgaaggacgggggccccgggcgcggcggctgcgacaacccgggcaagttccaccacgtggagaagagcgcgtcgtgcgcgccgctctgcacgcccggcgtggacgtgtactggagccgcgaggacaagcgcttcgcagtggtctggctggccatctgggcggtgctgtgcttcttctccagcgccttcaccgtgctcaccttcctcatcgacccggcccgcttccgctaccccgagcgccccatcatcttcctctccatgtgctactgcgtctactccgtgggctacctcatccgcctcttcgccggcgccgagagcatcgcctgcgaccgggacagcggccagctctatgtcatccaggagggactggagagcaccggctgcacgctggtcttcctggtcctctactacttcggcatggccagctcgctgtggtgggtggtcctcacgctcacctggttcctggccgccggcaagaagtggggccacgaggccatcgaagccaacagcagctacttccacctggcagcctgggccatcccggcggtgaagaccatcctgatcctggtcatgcgcagggtggcgggggacgagctcaccggggtctgctacgtgggcagcatggacgtcaacgcgctcaccggcttcgtgctcattcccctggcctgctacctggtcatcggcacgtccttcatcctctcgggcttcgtggccctgttccacatccggagggtgatgaagacgggcggcgagaacacggacaagctggagaagctcatggtgcgtatcgggctcttctctgtgctgtacaccgtgccggccacctgtgtgatcgcctgctacttttacgaacgcctcaacatggattactggaagatcctggcggcgcagcacaagtgcaaaatgaacaaccagactaaaacgctggactgcctgatggccgcctccatccccgccgtggagatcttcatggtgaagatctttatgctgctggtggtggggatcaccagcgggatgtggatttggacctccaagactctgcagtcctggcagcaggtgtgcagccgtaggttaaagaagaagagccggagaaaaccggccagcgtgatcaccagcggtgggatttacaaaaaagcccagcatccccagaaaactcaccacgggaaatatgagatccctgcccagtcgcccacctgcgtgtga

SEQ ID NO: 25 - protein, 581 aa, synthetic construct
  REGION 1..581: note = synthetic
MQRPGPRLWLVLQVMGSCAAISSMDMERPGDGKCQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLPNKNDPNYLCMEAPNNGSDEPTRGSGLFPPLFRPQRPHSAQEHPLKDGGPGRGGCDNPGKFHHVEKSASCAPLCTPGVDVYWSREDKRFAVVWLAIWAVLCFFSSAFTVLTFLIDPARFRYPERPIIFLSMCYCVYSVGYLIRLFAGAESIACDRDSGQLYVIQEGLESTGCTLVFLVLYYFGMASSLWWVVLTLTWFLAAGKKWGHEAIEANSSYFHLAAWAIPAVKTILILVMRRVAGDELTGVCYVGSMDVNALTGFVLIPLACYLVIGTSFILSGFVALFHIRRVMKTGGENTDKLEKLMVRIGLFSVLYTVPATCVIACYFYERLNMDYWKILAAQHKCKMNNQTKTLDCLMAASIPAVEIFMVKIFMLLVVGITSGMWIWTSKTLQSWQQVCSRRLKKKSRRKPASVITSGGIYKKAQHPQKTHHGKYEIPAQSPTCV

SEQ ID NO: 26 - DNA, 2295 bp, synthetic construct
  misc_feature 1..2295: note = synthetic
  source 1..2295: mol_type = other DNA
atgaggccggcggacttgctgcagctggtgctgctgctcgacctgcccagggacctgggcggaatggggtgttcgtctccaccctgcgagtgccatcaggaggaggacttcagagtcacctgcaaggatattcaacgcatccccagcttaccgcccagtacgcagactctgaagcttattgagactcacctgagaactattccaagtcatgcattttctaatctgcccaatatttccagaatctacgtatctatagatgtgactctgcagcagctggaatcacactccttctacaatttgagtaaagtgactcacatagaaattcggaataccaggaacttaacttacatagaccctgatgccctcaaagagctccccctcctaaagttccttggcattttcaacactggacttaaaatgttccctgacctgaccaaagtttattccactgatatattctttatacttgaaattacagacaacccttacatgacgtcaatccctgtgaatgcttttcagggactatgcaatgaaaccttgacactgaagctgtacaacaatggctttacttcagtccaaggatatgctttcaatgggacaaagctggatgctgtttacctaaacaagaataaatacctgacagttattgacaaagatgcatttggaggagtatacagtggaccaagcttgctggacgtgtctcaaaccagtgtcactgcccttccatccaaaggcctggagcacctgaaggaactgatagcaagaaacacctggactcttaagaaacttccactttccttgagtttccttcacctcacacgggctgacctttcttacccaagccactgctgtgcttttaagaatcagaagaaaatcagaggaatccttgagtccttgatgtgtaatgagagcagtatgcagagcttgcgccagagaaaatctgtgaatgccttgaatagccccctccaccaggaatatgaagagaatctgggtgacagcattgttgggtacaaggaaaagtccaagttccaggatactcataacaacgctcattattacgtcttctttgaagaacaagaggatgagatcattggttttggccaggagctcaaaaacccccaggaagagactctacaagcttttgacagccattatgactacaccatatgtggggacagtgaagacatggtgtgtacccccaagtccgatgagttcaacccgtgtgaagacataatgggctacaagttcctgagaattgtggtgtggttcgttagtctgctggctctcctgggcaatgtctttgtcctgcttattctcctcaccagccactacaaactgaacgtcccccgctttctcatgtgcaacctggcctttgcggatttctgcatggggatgtacctgctcctcatcgcctctgtagacctctacactcactctgagtactacaaccatgccatcgactggcagacaggccctgggtgcaacacggctggtttcttcactgtctttgcaagcgagttatcggtgtatacgctgacggtcatcaccctggagcgctggtatgccatcaccttcgccatgcgcctggaccggaagatccgcctcaggcacgcatgtgccatcatggttgggggctgggtttgctgcttccttctcgccctgcttcctttggtgggaataagtagctatgccaaagtcagtatctgcctgcccatggacaccgagacccctcttgctctggcatatattgtttttgttctgacgctcaacatagttgccttcgtcatcgtctgctgctgttatgtgaagatctacatcacagtccgaaatccgcagtacaacccaggggacaaagataccaaaattgccaagaggatggctgtgttgatcttcaccgacttcatatgcatggccccaatctcattctatgctctgtcagcaattctgaacaagcctctcatcactgttagcaactccaaaatcttgctggtactcttctatccacttaactcctgtgccaatccattcctctatgctattttcaccaaggccttccagagggatgtgttcatcctactcagcaagtttggcatctgtaaacgccaggctcaggcataccgggggcagagggttcctccaaagaacagcactgatattcaggttcaaaaggttacccacgagatgaggcagggtctccacaacatggaagatgtctatgaactgattgaaaactcccatctaaccccaaagaagcaaggccaaatctcagaagagtatatgcaaacggttttgtaa

SEQ ID NO: 27 - protein, 764 aa, synthetic construct
  REGION 1..764: note = synthetic
MRPADLLQLVLLLDLPRDLGGMGCSSPPCECHQEEDFRVTCKDIQRIPSLPPSTQTLKLIETHLRTIPSHAFSNLPNISRIYVSIDVTLQQLESHSFYNLSKVTHIEIRNTRNLTYIDPDALKELPLLKFLGIFNTGLKMFPDLTKVYSTDIFFILEITDNPYMTSIPVNAFQGLCNETLTLKLYNNGFTSVQGYAFNGTKLDAVYLNKNKYLTVIDKDAFGGVYSGPSLLDVSQTSVTALPSKGLEHLKELIARNTWTLKKLPLSLSFLHLTRADLSYPSHCCAFKNQKKIRGILESLMCNESSMQSLRQRKSVNALNSPLHQEYEENLGDSIVGYKEKSKFQDTHNNAHYYVFFEEQEDEIIGFGQELKNPQEETLQAFDSHYDYTICGDSEDMVCTPKSDEFNPCEDIMGYKFLRIVVWFVSLLALLGNVFVLLILLTSHYKLNVPRFLMCNLAFADFCMGMYLLLIASVDLYTHSEYYNHAIDWQTGPGCNTAGFFTVFASELSVYTLTVITLERWYAITFAMRLDRKIRLRHACAIMVGGWVCCFLLALLPLVGISSYAKVSICLPMDTETPLALAYIVFVLTLNIVAFVIVCCCYVKIYITVRNPQYNPGDKDTKIAKRMAVLIFTDFICMAPISFYALSAILNKPLITVSNSKILLVLFYPLNSCANPFLYAIFTKAFQRDVFILLSKFGICKRQAQAYRGQRVPPKNSTDIQVQKVTHEMRQGLHNMEDVYELIENSHLTPKKQGQISEEYMQTVL

SEQ ID NO: 28 - DNA, 1869 bp, synthetic construct
  misc_feature 1..1869: note = synthetic
  source 1..1869: mol_type = other DNA
atgaaggaaaatgtggcatctgcaaccgttttcactctgctactttttctcaacacctgccttctgaatggacagttacctcctggaaaacctgagatctttaaatgtcgttctcccaataaggaaacattcacctgctggtggaggcctgggacagatggaggacttcctaccaattattcactgacttaccacagggaaggagagacactcatgcatgaatgtccagactacataaccggtggccccaactcctgccactttggcaagcagtacacctccatgtggaggacatacatcatgatggtcaatgccactaaccagatgggaagcagtttctcggatgaactttatgtggacgtgacttacatagttcagccagaccctcctttggagctggctgtggaagtaaaacagccagaagacagaaaaccctacctgtggattaaatggtctccacctaccctgattgacttaaaaactggttggttcacgctcctgtatgaaattcgattaaaacccgagaaagcagctgagtgggagatccattttgctgggcagcaaacagagtttaagattctcagcctacatccaggacagaaataccttgtccaggttcgctgcaaaccagaccatggatactggagtgcatggagtccagcgaccttcattcagatacctagtgacttcaccatgaatgatacaaccgtgtggatctctgtggctgtcctttctgctgtcatctgtttgattattgtctgggcagtggctttgaagggctatagcatggtgacctgcatctttccgccagttcctgggccaaaaataaaaggatttgatgctcatctgttggagaagggcaagtctgaagaactactgagtgccttgggatgccaagactttcctcccacttctgactatgaggacttgctggtggagtatttagaagtagatgatagtgaggaccagcatctaatgtcagtccattcaaaagaacacccaagtcaaggtatgaaacccacatacctggatcctgacactgactcaggccgggggagctgtgacagcccttcccttttgtctgaaaagtgtgaggaaccccaggccaatccctccacattctatgatcctgaggtcattgagaagccagagaatcctgaaacaacccacacctgggacccccagtgcataagcatggaaggcaaaatcccctattttcatgctggtggatccaaatgttcaacatggcccttaccacagcccagccagcacaaccccagatcctcttaccacaatattactgatgtgtgtgagctggctgtgggccctgcaggtgcaccggccactctgttgaatgaagcaggtaaagatgctttaaaatcctctcaaaccattaagtctagagaagagggaaaggcaacccagcagagggaggtagaaagcttccattctgagactgaccaggatacgccctggctgctgccccaggagaaaaccccctttggctccgctaaacccttggattatgtggagattcacaaggtcaacaaagatggtgcattatcattgctaccaaaacagagagagaacagcggcaagcccaagaagcccgggactcctgagaacaataaggagtatgccaaggtgtccggggtcatggataacaacatcctggtgttggtgccagatccacatgctaaaaacgtggcttgctttgaagaatcagccaaagaggccccaccatcacttgaacagaatcaagctgagaaagccctggccaacttcactgcaacatcaagcaagtgcaggctccagctgggtggtttggattacctggatcccgcatgttttacacactcctttcactga

SEQ ID NO: 29 - protein, 622 aa, synthetic construct
  REGION 1..622: note = synthetic
MKENVASATVFTLLLFLNTCLLNGQLPPGKPEIFKCRSPNKETFTCWWRPGTDGGLPTNYSLTYHREGETLMHECPDYITGGPNSCHFGKQYTSMWRTYIMMVNATNQMGSSFSDELYVDVTYIVQPDPPLELAVEVKQPEDRKPYLWIKWSPPTLIDLKTGWFTLLYEIRLKPEKAAEWEIHFAGQQTEFKILSLHPGQKYLVQVRCKPDHGYWSAWSPATFIQIPSDFTMNDTTVWISVAVLSAVICLIIVWAVALKGYSMVTCIFPPVPGPKIKGFDAHLLEKGKSEELLSALGCQDFPPTSDYEDLLVEYLEVDDSEDQHLMSVHSKEHPSQGMKPTYLDPDTDSGRGSCDSPSLLSEKCEEPQANPSTFYDPEVIEKPENPETTHTWDPQCISMEGKIPYFHAGGSKCSTWPLPQPSQHNPRSSYHNITDVCELAVGPAGAPATLLNEAGKDALKSSQTIKSREEGKATQQREVESFHSETDQDTPWLLPQEKTPFGSAKPLDYVEIHKVNKDGALSLLPKQRENSGKPKKPGTPENNKEYAKVSGVMDNNILVLVPDPHAKNVACFEESAKEAPPSLEQNQAEKALANFTATSSKCRLQLGGLDYLDPACFTHSFH

SEQ ID NO: 30 - DNA, 1212 bp, synthetic construct
  misc_feature 1..1212: note = synthetic
  source 1..1212: mol_type = other DNA
atgcctaggccaggcacaatggctctgtgcctgctgaccctggtgctgtccctgctgccacctcaggctgctgccacaaacaccacaatcaagtccaaccccacatctaccccaacagtgcccaggaccacaacctgcttcggcgatggatgtcagaacacagcttctagatgcaagaacggcggaacctgggacggcctgaagtgccagtgtcctaacctgtactacggagagctgtgcgaggaggtggtgagctccatcgatatcggcccacccgagacaatctctgcccagatggagctgaccgtgacagtgaccagcgtgaagttcaccgaggagctgaagaaccactctagccaggagttccaggagtttaagcagaccttcaccgagcagatgaacatcgtgtactctggcatccctgagtacgtgggagtgaacatcaccaagctgcggctgggaagcgtggtggtggagcacgatgtgctgctgcgcacaaagtacaccccagagtacaagacagtgctggacaacgctaccgaggtggtgaaggagaagatcacaaaggtgacaacccagcagatcatgatcaacgatatctgcagcgacatgatgtgcttcaacacaaccggaacccaggtgcagaacatcacagtgacccagtacgatccagaggaggactgccggaagatggccaaggagtacggcgattacttcgtggtggagtaccgcgaccagaagccctactgcatctccccttgtgagccaggattttccgtgtctaagaactgcaacctgggcaagtgtcagatgagcctgtccggaccacagtgcctgtgcgtgacaaccgagacacactggtactccggcgagacatgtaaccagggaacccagaagtctctggtgtacggcctggtgggcgctggagtggtgctgatgctgatcatcctggtggccctgctgatgctggtgtttaggtctaagagagaggtgaagaggcagaagtacagactgagccagctgtacaagtggcaggaggaggattccggaccagctcctggaacattccagaacatcggatttgacatctgtcaggacgatgacagcatccacctggagtctatctacagcaacttccagccctccctgagacacatcgaccctgagacaaagatccggatccagcgccctcaggtcatgacaacctccttt

SEQ ID NO: 31 - protein, 404 aa, synthetic construct
  REGION 1..404: note = synthetic
MPRPGTMALCLLTLVLSLLPPQAAATNTTIKSNPTSTPTVPRTTTCFGDGCQNTASRCKNGGTWDGLKCQCPNLYYGELCEEVVSSIDIGPPETISAQMELTVTVTSVKFTEELKNHSSQEFQEFKQTFTEQMNIVYSGIPEYVGVNITKLRLGSVVVEHDVLLRTKYTPEYKTVLDNATEVVKEKITKVTTQQIMINDICSDMMCFNTTGTQVQNITVTQYDPEEDCRKMAKEYGDYFVVEYRDQKPYCISPCEPGFSVSKNCNLGKCQMSLSGPQCLCVTTETHWYSGETCNQGTQKSLVYGLVGAGVVLMLIILVALLMLVFRSKREVKRQKYRLSQLYKWQEEDSGPAPGTFQNIGFDICQDDDSIHLESIYSNFQPSLRHIDPETKIRIQRPQVMTTSF

SEQ ID NO: 32 - DNA, 3222 bp, synthetic construct
  misc_feature 1..3222: note = synthetic
  source 1..3222: mol_type = other DNA
atgaagacgttgctgttggacttggctttgtggtcactgctcttccagcccgggtggctgtcctttagttcccaggtgagtcagaactgccacaatggcagctatgaaatcagcgtcctgatgatgggcaactcagcctttgcagagcccctgaaaaacttggaagatgcggtgaatgaggggctggaaatagtgagaggacgtctgcaaaatgctggcctaaatgtgactgtgaacgctactttcatgtattcggatggtctgattcataactcaggcgactgccggagtagcacctgtgaaggcctcgacctactcaggaaaatttcaaatgcacaacggatgggctgtgtcctcatagggccctcatgtacatactccaccttccagatgtaccttgacacagaattgagctaccccatgatctcagctggaagttttggattgtcatgtgactataaagaaaccttaaccaggctgatgtctccagctagaaagttgatgtacttcttggttaacttttggaaaaccaacgatctgcccttcaaaacttattcctggagcacttcgtatgtttacaagaatggtacagaaactgaggactgtttctggtaccttaatgctctggaggctagcgtttcctatttctcccacgaactcggctttaaggtggtgttaagacaagataaggagtttcaggatatcttaatggaccacaacaggaaaagcaatgtgattattatgtgtggtggtccagagttcctctacaagctgaagggtgaccgagcagtggctgaagacattgtcattattctagtggatcttttcaatgaccagtactttgaggacaatgtcacagcccctgactatatgaaaaatgtccttgttctgacgctgtctcctgggaattcccttctaaatagctctttctccaggaatctatcaccaacaaaacgagactttgctcttgcctatttgaatggaatcctgctctttggacatatgctgaagatatttcttgaaaatggagaaaatattaccacccccaaatttgctcatgctttcaggaatctcacttttgaagggtatgacggtccagtgaccttggatgactggggggatgttgacagtaccatggtgcttctgtatacctctgtggacaccaagaaatacaaggttcttttgacctatgatacccacgtaaataagacctatcctgtggatatgagccccacattcacttggaagaactctaaacttcctaatgatattacaggccggggccctcagatcctgatgattgcagtcttcaccctcactggagctgtggtgctgctcctgctcgtcgctctcctgatgctcagaaaatatagaaaagattatgaacttcgtcagaaaaaatggtcccacattcctcctgaaaatatctttcctctggagaccaatgagaccaatcatgttagcctcaagatcgatgatgacaaaagacgagatacaatccagagactacgacagtgcaaatacgacaaaaagcgagtgattctcaaagatctcaagcacaatgatggtaatttcactgaaaaacagaagatagaattgaacaagttgcttcagattgactattacaacctgaccaagttctacggcacagtgaaacttgataccatgatcttcggggtgatagaatactgtgagagaggatccctccgggaagttttaaatgacacaatttcctaccctgatggcacattcatggattgggagtttaagatctctgtcttgtatgacattgctaagggaatgtcatatctgcactccagtaagacagaagtccatggtcgtctgaaatctaccaactgcgtagtggacagtagaatggtggtgaagatcactgattttggctgcaattccattttacctccaaaaaaggacctgtggacagctccagagcacctccgccaagccaacatctctcagaaaggagatgtgtacagctatgggatcatcgcacaggagatcatcctgcggaaagaaaccttctacactttgagctgtcgggaccggaatgagaagattttcagagtggaaaattccaatggaatgaaacccttccgcccagatttattcttggaaacagcagaggaaaaagagctagaagtgtacctacttgtaaaaaactgttgggaggaagatccagaaaagagaccagatttcaaaaaaattgagactacacttgccaagatatttggactttttcatgaccaaaaaaatgaaagctatatggataccttgatccgacgtctacagctatattctcgaaacctggaacatctggtagaggaaaggacacagctgtacaaggcagagagggacagggctgacagacttaactttatgttgcttccaaggctagtggtaaagtctctgaaggagaaaggctttgtggagccggaactatatgaggaagttacaatctacttcagtgacattgtaggtttcactactatctgcaaatacagcacccccatggaagtggtggacatgcttaatgacatctataagagttttgaccacattgttgatcatcatgatgtctacaaggtggaaaccatcggtgatgcgtacatggtggctagtggtttgcctaagagaaatggcaatcggcatgcaatagacattgccaagatggccttggaaatcctcagcttcatggggacctttgagctggagcatcttcctggcctcccaatatggattcgcattggagttcactctggtccctgtgctgctggagttgtgggaatcaagatgcctcgttattgtctatttggagatacggtcaacacagcctctaggatggaatccactggcctccctttgagaattcacgtgagtggctccaccatagccatcctgaagagaactgagtgccagttcctttatgaagtgagaggagaaacatacttaaagggaagaggaaatgagactacctactggctgactgggatgaaggaccagaaattcaacctgccaacccctcctactgtggagaatcaacagcgtttgcaagcagaattttcagacatgattgccaactctttacagaaaagacaggcagcagggataagaagccaaaaacccagacgggtagccagctataaaaaaggcactctggaatacttgcagctgaataccacagacaaggagagcacctatttttaa

SEQ ID NO: 33 - protein, 1073 aa, synthetic construct
  REGION 1..1073: note = synthetic
MKTLLLDLALWSLLFQPGWLSFSSQVSQNCHNGSYEISVLMMGNSAFAEPLKNLEDAVNEGLEIVRGRLQNAGLNVTVNATFMYSDGLIHNSGDCRSSTCEGLDLLRKISNAQRMGCVLIGPSCTYSTFQMYLDTELSYPMISAGSFGLSCDYKETLTRLMSPARKLMYFLVNFWKTNDLPFKTYSWSTSYVYKNGTETEDCFWYLNALEASVSYFSHELGFKVVLRQDKEFQDILMDHNRKSNVIIMCGGPEFLYKLKGDRAVAEDIVIILVDLFNDQYFEDNVTAPDYMKNVLVLTLSPGNSLLNSSFSRNLSPTKRDFALAYLNGILLFGHMLKIFLENGENITTPKFAHAFRNLTFEGYDGPVTLDDWGDVDSTMVLLYTSVDTKKYKVLLTYDTHVNKTYPVDMSPTFTWKNSKLPNDITGRGPQILMIAVFTLTGAVVLLLLVALLMLRKYRKDYELRQKKWSHIPPENIFPLETNETNHVSLKIDDDKRRDTIQRLRQCKYDKKRVILKDLKHNDGNFTEKQKIELNKLLQIDYYNLTKFYGTVKLDTMIFGVIEYCERGSLREVLNDTISYPDGTFMDWEFKISVLYDIAKGMSYLHSSKTEVHGRLKSTNCVVDSRMVVKITDFGCNSILPPKKDLWTAPEHLRQANISQKGDVYSYGIIAQEIILRKETFYTLSCRDRNEKIFRVENSNGMKPFRPDLFLETAEEKELEVYLLVKNCWEEDPEKRPDFKKIETTLAKIFGLFHDQKNESYMDTLIRRLQLYSRNLEHLVEERTQLYKAERDRADRLNFMLLPRLVVKSLKEKGFVEPELYEEVTIYFSDIVGFTTICKYSTPMEVVDMLNDIYKSFDHIVDHHDVYKVETIGDAYMVASGLPKRNGNRHAIDIAKMALEILSFMGTFELEHLPGLPIWIRIGVHSGPCAAGVVGIKMPRYCLFGDTVNTASRMESTGLPLRIHVSGSTIAILKRTECQFLYEVRGETYLKGRGNETTYWLTGMKDQKFNLPTPPTVENQQRLQAEFSDMIANSLQKRQAAGIRSQKPRRVASYKKGTLEYLQLNTTDKESTYF

SEQ ID NO: 34 - DNA, 987 bp, synthetic construct
  misc_feature 1..987: note = synthetic
  source 1..987: mol_type = other DNA
atgactgtggagaaggaggcccctgatgcgcacttcactgtggacaaacagaacatctccctctggccccgagagcctcctcccaagtccggtccatctctggtcccggggaaaacacccacagtccgtgctgcattaatctgcctgacgctggtcctggtcgcctccgtcctgctgcaggccgtcctttatccccggtttatgggcaccatatcagatgtaaagaccaatgtccagttgctgaaaggtcgtgtggacaacatcagcaccctggattctgaaattaaaaagaatagtgacggcatggaggcagctggcgttcagatccagatggtgaatgagagcctgggttatgtgcgttctcagttcctgaagttaaaaaccagtgtggagaaggccaacgcacagatccagatcttaacaagaagttgggaagaagtcagtaccttaaatgcccaaatcccagagttaaaaagtgatttggagaaagccagtgctttaaatacaaagatccgggcactccagggcagcttggagaatatgagcaagttgctcaaacgacaaaatgatattctacaggtggtttctcaaggctggaagtacttcaaggggaacttctattacttttctctcattccaaagacctggtatagtgccgagcagttctgtgtgtccaggaattcacacctgacctcggtgacctcagagagtgagcaggagtttctgtataaaacagcggggggactcatctactggattggcctgactaaagcagggatggaaggggactggtcctgggtggatgacacgccattcaacaaggtccaaagtgtgaggttctggattccaggtgagcccaacaatgctgggaacaatgaacactgtggcaatataaaggctccctcacttcaggcctggaatgatgccccatgtgacaaaacgtttcttttcatttgtaagcgaccctatgtcccatcagaaccgtga

SEQ ID NO: 35 - protein, 328 aa, synthetic construct
  REGION 1..328: note = synthetic
MTVEKEAPDAHFTVDKQNISLWPREPPPKSGPSLVPGKTPTVRAALICLTLVLVASVLLQAVLYPRFMGTISDVKTNVQLLKGRVDNISTLDSEIKKNSDGMEAAGVQIQMVNESLGYVRSQFLKLKTSVEKANAQIQILTRSWEEVSTLNAQIPELKSDLEKASALNTKIRALQGSLENMSKLLKRQNDILQVVSQGWKYFKGNFYYFSLIPKTWYSAEQFCVSRNSHLTSVTSESEQEFLYKTAGGLIYWIGLTKAGMEGDWSWVDDTPFNKVQSVRFWIPGEPNNAGNNEHCGNIKAPSLQAWNDAPCDKTFLFICKRPYVPSEP

SEQ ID NO: 36 - protein, 480 aa, synthetic construct
  REGION 1..480: note = synthetic
MALPVTALLLPLALLLHAARPDIQMTQSPASLSVSVGETVTITCRASENIYSNLAWYQQKQGKSPQLLVYVATNLADGVPSRFSGSGSGTQYSLKINSLQSEDFGSYYCQHFWGTPYTFGGGTKLGGGSGGGGSGGGGSEVQLQQSGAELVKPGASVKLSCTASGFNINDTYMHWVKQRPEQGLEWIGRIDPANGNTKYDPKFQGKATITADTSSNTAYLQLSSLTSEDTAVYYCARGARGSRFAYWGQGTLVTVSAAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 37 - protein, 488 aa, synthetic construct
  REGION 1..488: note = synthetic
MALPVTALLLPLALLLHAARPQSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVLGGGGSGGGGSGGGGSEVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 496 aa, synthetic construct
  REGION 1..496: note = synthetic
MALPVTALLLPLALLLHAARPDIVLTQSPASLAVSLGQGATISCRASKSVSTSGYTYMHWYQQKPGQPPKLLIYLASNLESGVPARFSGSGSGTDFTLNIHPVEEEDAATYYCQHSGELPPSFGGGTKLEIKRAGGGGSGGGGSGGGGSEVQLVESGGDLVKPGGSLKLSCAVSGFTFSSYGMSWVRQTPDKRLEWVATVSSGGTYTYYPDSVKGRFTISRDNAKNTLYLQMSSLKSEDSAMYYCARHRGNYYATYYYAMDYWGQGTSVTVSSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 487 aa, synthetic construct
  REGION 1..487: note = synthetic
MALPVTALLLPLALLLHAARPDVQITQSPSYLAASPGETITINCRASKSISKYLAWYQEKPGKTNKLLIYSGSTLQSGIPSRFSGSGSGTDFTLTISSLEPEDFAMYYCQQHHEYPYTFGGGTKLEIKGGGGSGGGGSGGGGSQVQLQQPGAELVRPGASVKLSCKASGYTFTTYWMNWVKQRPEQGLEWIGRIDPYDSETHYNQKFKDKAILTVDKSSSTAYMQLSSLTSEDSAVYYCARGPYYGTNPWFPYWGQGTLVTVSSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 485 aa, synthetic construct
  REGION 1..485: note = synthetic
MALPVTALLLPLALLLHAARPEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 41 - protein, 490 aa, synthetic construct
  REGION 1..490: note = synthetic
MALPVTALLLPLALLLHAARPDVVMTQTPLSLPVRLGDQASISCRSSQSLVHSNGNTYLHWYLQKPGQSPKLLIYKVSNRFSGVPDRFSGSGSGTNFTLKISRVEAEDLGLYFCSQSTHVPYTFGGGTKLEIKRAGGGGSGGGGSGGGGSQVQLRQSGPELVKPGASVKMSCKASGYTFTDYVISWVKQRTGQGLEWIGDIYPGSGYSFYNENFKGKATLTADKSSTTAYMQLSSLTSEDSAVYFCATYYNYPFAYWGQGTLVTVSAAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 42 - protein, 443 aa, synthetic construct
  REGION 1..443: note = synthetic
MALPVTALLLPLALLLHAARPLPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNCAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 43 - protein, 434 aa, synthetic construct
  REGION 1..434: note = synthetic
MALPVTALLLPLALLLHAARPRCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLKCRIIHNNNCAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - protein, 190 aa, synthetic construct
  REGION 1..190: note = synthetic
RCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLKCRIIHNNNC

SEQ ID NO: 45 - protein, 69 aa, synthetic construct
  REGION 1..69: note = Synthesis
TTTPAPRPPTPAPTIASQPLSLRPEACRPAAGGAVHTRGLDFACDIYIWAPLAGTCGVLLLSLVITLYC